(19) United States
(12) Patent Application Publication
(10) Pub. No.: US 2026/0086088 A1
(43) Pub. Date: Mar. 26, 2026

(54) BIOINKS, METHODS OF PRINTING AND RELATED USES

(71) Applicant: McMaster University, Hamilton (CA)

(72) Inventors: Tohid F. Didar, Dundas (CA); Zeinab Hosseinidoust, Dundas (CA); Amid Shakeri, Richmond Hill (CA); Lubna Najm, Hamilton (CA)

(21) Appl. No.: 19/335,980

(22) Filed: Sep. 22, 2025

Related U.S. Application Data
(60) Provisional application No. 63/696,904, filed on Sep. 20, 2024.

(51) Int. Cl. G01N 33/543; C09D 11/04; C09D 11/14

(52) U.S. Cl. CPC G01N 33/54386; C09D 11/04; C09D 11/14

(57) ABSTRACT

A pair of bioinks for printing a biosensor comprise: a first bioink being free of a crosslinking agent and comprising a scaffolding moiety and a biorecognition element for printing on a substrate; and a second bioink comprising a crosslinking agent for printing on and crosslinking the first bioink to form the biosensor.

### CROSS-REFERENCE TO RELATED APPLICATION

This application claims priority to and the benefit of U.S. Provisional Patent Application No. 63/696,904, filed Sep. 20, 2024, the content of which is hereby incorporated herein by reference in its entirety.

### FIELD

The present disclosure relates to the field of bioprinting, and, in particular, to bioinks, methods of printing and uses thereof.

### BACKGROUND

Diagnostic technologies rely on highly sensitive and reproducible detection of biomarkers in complex fluids (1,2). This necessitates biosensors that provide a high surface area for biorecognition, while simultaneously remaining stable when in contact with clinical samples. Otherwise, the fluidic nature and complex composition of such samples can non-specifically interact with the bio interface, reducing sensing integrity (3,4). Immunofluorescence assays (IFAs) have been widely researched for multiple decades and are now regularly used for biosensing technology (4-6). These assays generally employ sensing interfaces comprised of two-dimensional (2D) microarrays of biorecognition molecules (4). Microarray production enables accurate multiplex detection of different biomarkers in parallel, through the employment of highly specific protein-protein biorecognition cascades. Such systems operate well within complex clinical samples that contain various cells and proteins naturally present in biological fluids (7).

Traditional antibody microarray fabrication processes involve the selection of antibodies, surface preparation, immobilization of antibodies, blocking, sample incubation, and detection (4). The sensitivity of these IFA-based platforms is strongly tied to the geometric topography of the microarrays, such as biomolecule density, structure, and surface area of the immobilized capture antibodies (CAbs) (8-14). Often, to achieve clinically relevant limits of detection (LODs), biosensing microarrays must typically have LODs in the picolitre or sub-picolitre range for most diseases (15-20). The additional development of microfluidic chips, lateral flow assays or bead-based assays is required for conventional 2D microarray systems. This results in highly complex fabrication protocols and mechanical properties that are difficult to translate to high throughput and industry scale manufacturing (21-25). As such, while most biosensors reported in literature are created with 2D microarrays, recent advances in biosensor design have focused on increasing biomolecule density through the introduction of hierarchy. This has enhanced sensor sensitivity via signal amplification (26). Manufacturing of hierarchical surfaces, however, is also often time-consuming and incompatible with high throughput fabrication (8,26).

A more functional approach is creating three-dimensional (3D) hydrogel-based microarrays with high bio-functional surface area (27,28). As an additional advantage, the hydrogel microstructure retains high water content, which keeps antibodies hydrated throughout the fabrication procedure (29). There have been various efforts to integrate hydrogel microarrays for biosensing applications. For example, Li et al. demonstrated the use of inkjet printing to fabricate multi-biosensors based on nanostructured conductive hydrogels (30). Additionally, some studies have examined UV-crosslinkable microarrays using poly(ethylene glycol) diacrylate (PEGDA)-based hydrogels (31) or N,N-dimethylacrylamide (DMAA)-based hydrogels (32,33), where the hydrogels are mixed with capture probes and photoinitiators. However, these systems often involve more complex fabrication processes and have not achieved the sufficiently low sensitivity and wide range of linearity needed for effective detection.

Noncontact dispensing is a standard technique for the fabrication of microarray systems. Here, printers that utilize piezo-driven injection systems create microarrays of biomolecules in a noncontact fashion with high precision and accuracy, which limits the risk of damaging sensitive solutions and substrates (29,34). These printers have already been incorporated in many production lines for the high throughput fabrication of biomolecular microarrays (7,27,28,35,36). However, implementation of noncontact printers for hydrogel printing is very challenging because their viscous nature presents a high risk of nozzle blockage (35). Therefore, there is a need for bioinks and printing methods for microarray systems that do not cause nozzle blockage.

The background herein is included solely to explain the context of the disclosure. This is not to be taken as an admission that any of the material referred to was published, known, or part of the common general knowledge as of the priority date.

### SUMMARY

The present disclosure describes a platform for three-dimensional microarray bioprinting, wherein a two-step printing approach enables the high-throughput fabrication of immunosorbent hydrogels. The bioink printed first was composed entirely of proteins and clinically relevant capture antibodies, while another bioink containing a crosslinking agent, is printed directly on top. This technique differs from other reported noncontact printing methods, which typically optimize a single bioink for printing (35-41). With this noncontact printing approach, protein cross-linking was promoted on the substrate rather than inside the printer's nozzle, resulting in hydrogel formation on the substrate, preventing nozzle blockage. This enabled the development of 3D hydrogel microarrays with biosensing capabilities. Compared to two-dimensional microarrays, these proteinaceous microarrays offer 3-fold increases in signal intensity. When tested with clinically relevant biomarkers, ultrasensitive single plex, and multiplex detection of interleukin-6 (IL-6) (LOD 0.3 pg/mL) and tumor necrosis factor receptor 1 (TNF R1) (LOD 1 pg/mL) is observed. When challenged with clinical samples, these hydrogel microarrays consistently discern elevated interleukin-6 levels in blood plasma derived from patients with systemic blood infections. This present invention demonstrates an easy to implement, high-throughput fabrication and ultrasensitive detection, using these three-dimensional microarrays which will enable better clinical monitoring of disease progression, yielding improved patient outcomes.

In accordance with an aspect, there is provided a pair of bioinks for printing a biosensor:
a first bioink being free of a crosslinking agent and comprising a scaffolding moiety and a biorecognition element for printing on a substrate; and
a second bioink comprising a crosslinking agent for printing on and crosslinking the first bioink to form the biosensor.

In an aspect, the scaffolding moiety comprises BSA, collagen, gelatin, alginate, chitosan, or any combination thereof.

In an aspect, the scaffolding moiety is present in an amount sufficient to achieve consistency in the morphology and circularity of the printed biosensor.

In an aspect, the scaffolding moiety is present in an amount of less than about 2% by weight of the first bioink.

In an aspect, the biorecognition element comprises an antibody or fragment thereof, a peptide, a polynucleotide, an aptamer, an aptamer-conjugated nanoparticle, a DNAzyme, a bacteriophage, or any combination thereof.

In an aspect, the crosslinking agent comprises 1-ethyl-3-(3-dimethylaminopropyl) carbodiimide (EDC), N-hydroxysuccinimide (NHS), glutaraldehyde, N′,N′-dicyclohexyl carbodiimide (DCC), N,N′-diisopropyl carbodiimide (DIC), sulfo-NHS, thiophosgene, succinic anhydride, 3-mercaptopropyl trimethoxysilane (MPTMS), γ-maleimidobutyryloxy succinimide (GMBS), or any combination thereof.

In an aspect, the crosslinking agent is present in an amount sufficient to crosslink the first bioink such that the biosensor is capable of withstanding intense washing with water and/or assay buffer.

In an aspect, the crosslinking agent is present in an amount of about 2 mg/mL.

In an aspect, the substrate comprises glass, a thermoplastic substrate, cyclic olefin copolymer, polystyrene, polycarbonate, thermoset, polydimethylsiloxane, fabric, paper, cellulose, or any combination thereof.

In accordance with an aspect, there is provided a biosensor made from the pair of bioinks of described herein, wherein the first bioink is printed on the substrate and the second bioink is printed on the first bioink.

In an aspect, the biosensor comprises a plurality of layers of the first and second bioinks.

In an aspect, the biosensor comprises two or three layers of the first and second bioinks.

In accordance with an aspect, there is provided a microarray comprising a plurality of the biosensors described herein.

In an aspect, at least one of the biosensors comprises a first biorecognition element and at least one other of the biosensors comprises a second biorecognition element.

In accordance with an aspect, there is provided a method of printing a biosensor from the pair of bioinks described herein, the method comprising:
printing the first bioink onto the substrate; and
printing the second bioink onto the first bioink.

In an aspect, the method further comprises printing a second layer of the first bioink onto the second bioink followed by printing the second bioink onto the second layer of the first bioink.

In an aspect, the method further comprises an incubation step before printing the second layer.

In an aspect, the first bioink is printed from a first nozzle and the second bioink is printed from a second nozzle.

In accordance with an aspect, there is provided a hydrogel-based bioink comprising:
a) Bovine Serum Albumin (BSA)
b) One or more biorecognition elements
c) A cross-linking agent

In an embodiment, the bovine serum albumin (BSA) is in a concentration from 1-2%.

In an embodiment, the biorecognition element comprises one or more of capture antibodies, aptamers, aptamer-conjugated nanoparticles, DNAzymes.

In an embodiment, the crosslinking agent comprises one or more of 1-Ethyl-3-(3Dimethylaminopropyl) carbodiimide, glutaraldehyde, N′,N′-dicyclohexyl carbodiimide (DCC), N,N′-diisopropyl carbodiimide (DIC), EDC+N-Hydroxysuccinimide (NHS), EDC+sulfo-NHS, Thiophosgene, Succinic anhydride, 3-mercaptopropyl trimethoxysilane+γ-maleimidobutyryloxy succinimide (MPTMS+GMBS).

In a further embodiment, a method of printing the hydrogel-based bioink is provided, comprising:
a) Dispensing a primary bioink dot comprising BSA in phosphate-buffered saline (PBS) and one or more capture antibodies onto a substrate
b) Dispensing a secondary bioink comprising EDC diluted in 2-(N-morpholino) ethanesulfonic acid (MES) directly onto the primary bioink dot
c) Optionally, incubating for 30 minutes and adding one or more layers of the primary and secondary bioinks

In an embodiment, a piezo-driven injection system is used for printing the primary and secondary bioinks.

In an embodiment, the substrate comprises one of CO2 plasma-treated and fluorosilanized glass, thermoplastic substrates, cyclic olefin copolymer, polystyrene, polycarbonate, thermosets, polydimethylsiloxane, fabrics, papers, or cellulose based substrates.

In an embodiment, the hydrogel-based bioink is printed in a microarray pattern.

In an embodiment, the primary and secondary bioinks form a 3D biopolymer microstructure.

In an embodiment, the 3D microstructure of the bioinks increases the surface area for biosensing and demonstrates a limit of detection in a range of 0.3-1 pg/mL.

In an embodiment, a biosensor comprising the hydrogel-based bioink is provided.

In an embodiment, signal detection is measured by fluorescence, colorimetric, or electrochemical signals.

In an embodiment, the 3D hydrogel microarray provides multiplex detection of biomarkers through one or more biorecognition elements.

In an embodiment, use of the biosensor for detection of biomarkers in applications comprising disease diagnostics and/or monitoring, and the detection of bacteria, viruses, air pollution, fluid/water contamination, and food spoilage/contamination.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments but should be given the broadest interpretation consistent with the description as a whole.

### BRIEF DESCRIPTION OF THE FIGURES

Certain embodiments of the disclosure will now be described in greater detail with reference to the attached drawings in which:

FIG. 1 shows contact angle measurements and images of the plain untreated substrate compared to 30-minute and 3-hr fluorosilanized substrates in exemplary embodiments of the disclosure. Demonstrates where substrates increased in their hydrophobicity and contact angles after FS group deposition reached ˜111o. Error bars represent standard deviation based on 1-way ANOVA (or non-parametric/mixed Brown-Forsythe and Welch ANOVA model, p****<0.0001). Scale bar is 1 mm.

FIG. 2 shows experimental XPS data for the plain untreated glass substrate, with clear O1s and C1s peaks in exemplary embodiments of the disclosure.

FIG. 3 shows substrate preparation steps and XPS data in exemplary embodiments of the disclosure. (a) acetone sonication of glass slides, (b) CO2 plasma-treatment for 3 min to induce carboxyl groups, and (c) short fluorosilanization through CVD to increase surface hydrophobicity while the carboxyl groups are partially preserved. (d-f) Deconvoluted XPS results at C1s spectra of (d) glass substrates, (c) after CO2 plasma treatment, and (f) after partial fluorosilanization. (g) Surface fluorine percentage of glass substrates after each treatment step (p****<0.0001).

FIG. 4 shows experimental XPS data for the 3-min CO2 plasma-treated substrate, with a clear increase in O1s and decrease in C1s peaks compared to untreated glass in exemplary embodiments of the disclosure.

FIG. 5 shows experimental XPS data for the 30-min fluorosilanized substrate, with clear C1s, O1s and F1s peaks in exemplary embodiments of the disclosure.

FIG. 6 shows 3D microarray fabrication via noncontact printing of proteinaceous hydrogels, applied for IFA-based detection of proinflammatory biomarkers in exemplary embodiments of the disclosure. a) Schematic representation of the two-step noncontact printing of BSA bioink containing CAb, followed by printing of EDC solution directly on top, forming one layer of the hydrogel microstructure. After 30-minute incubation in humidity-controlled environment, the two-step print could be repeated to yield multiple layers that increase hydrogel volume. (b) Fluorescent image of 5×5 microarrays of printed 3D hydrogels. Scale bar is 500 μm. (c) Schematic of microdot components after printing bioink #1. (d) Crosslinking of hydrogel and covalent bonding to the carboxyl groups of the substrate upon the addition of bioink #2. (c) Confocal microscopy image of the 3D microstructure of the proteinaceous hydrogels. (f) Application of 3D hydrogels for detection IL-6 through IFA.

FIG. 7 shows proteinaceous hydrogels when only BSA is printed, where EDC is printed before BSA and when BSA is printed before EDC. Scale bar is 500 μm in exemplary embodiments of the disclosure.

FIG. 8 shows hydrogel microarrays with 3 mg/mL EDC printed before 1% BSA in exemplary embodiments of the disclosure. The inconsistency in the print may be due to the increased EDC concentration, which affects the nozzle's ability to generate stable droplets, as well as excessive salt formation from slight evaporation of the spot after the first print. Scale bar is 500 μm.

FIG. 9 shows optimization of BSA and EDC concentrations as well as number of layers in fabricating 3D hydrogel microarrays in exemplary embodiments of the disclosure. (a) Fluorescence images of 3D hydrogels containing FITC after the print, after a brief wash, and after an intense wash. Scale bar is 200 μm. The stability of the 3D hydrogels was significantly affected by the concentration of EDC. (b) Mean fluorescence intensities (MFIs) of the hydrogel microarrays printed with various concentrations of BSA, EDC, and number of layers, at different washing stages. Higher MFIs after the wash are associated with better stability of the 3D hydrogels. (c) signal-to-noise ratios (SNRs) calculated for all the different print parameters to assess non-specific attachment of IL-6 antibody containing hydrogels to streptavidin-Cy5. Overall, 2 L-1% BSA-2 mg/mL EDC conditions showed high stability and low SNR values. Error bars in (b) and (c) represents standard deviation (p*<0.05, p**<0.01, p***<0.001, p****<0.0001). (d) Heat Map graph of the SNR values. (c) Representative fluorescence images of 3D microarrays, showing the non-specific attachment of the IL-6 antibody containing hydrogels to streptavidin-Cy5 in different conditions. Scale bar is 200 μm.

FIG. 10 shows 3D hydrogels from stability analysis with 1% BSA, before wash, after brief wash and after intense wash in exemplary embodiments of the disclosure. Scale bar is 200 μm.

FIG. 11 shows 3D hydrogels from stability analysis with 1.5% BSA, before wash, after brief wash and after intense wash in exemplary embodiments of the disclosure. Scale bar is 200 μm.

FIG. 12 shows 3D hydrogels from stability analysis with 2% BSA, before wash, after brief wash and after intense wash in exemplary embodiments of the disclosure. Scale bar is 200 μm.

FIG. 13 shows topographical characterization of 3D hydrogels in exemplary embodiments of the disclosure. SEM images (i) and confocal images (ii and iii) of 3D hydrogel dots for (a) one-layer print, outlining the donut shape, (b) two-layer, depicting a smooth dome shape, and (c) three-layer print, delineating the pooling of excess hydrogel contents at the outermost borders. Scale bar of SEM images is 50 μm. Longitudinal scale coordinates indicate height (in μm) from the microscope's stage, including the heights of the substrates that the hydrogels were printed on and the stage accessory to mount the substrates. (d) Distribution of FITC (representing the CAb) throughout the hydrogel thickness, for 1 layer, 2 layers, and 3 layers of print. (e) Average thicknesses of each 3D hydrogel type versus the number of layers, and compares them through statistical analysis. Error bars represent standard deviation. (p****<0.0001). (f) Schematic representation of the structural shape and topographical distribution of hydrogel content upon printing each additional layer.

FIG. 14 shows 3D hydrogels from non-specific attachment analysis in exemplary embodiments of the disclosure. As EDC concentration increases, fluorescence from the hydrogels is observed, showing slight increase in non-specific attachment. Scale bar is 200 μm.

FIG. 15 shows MFIs of 3D hydrogels incubated overnight in 2% lysine diluted in PBS compared to 3D hydrogels without lysine incubation in exemplary embodiments of the disclosure. Error bars represent standard deviation (p****<0.0001).

FIG. 16 shows MFIs of 3D hydrogels created with tertiary prints of either APTES or 2% BSA solution diluted in PBS, compared to a control with no tertiary print in exemplary embodiments of the disclosure. Error bars represent standard deviation (p****<0.0001).

FIG. 17 shows MFIs of 3D hydrogels created with an additional print of NHS diluted in MES print compared to 3D hydrogels without NHS printed in exemplary embodiments of the disclosure. Error bars represent standard deviation (p*<0.05).

FIG. 18 shows MFI of 3D hydrogels printed with 5 drops compared to 10 drops in a microarray in exemplary embodiments of the disclosure. Error bars represent standard deviation (p****<0.0001).

FIG. 19 shows brightfield images of hydrogel microarrays in exemplary embodiments of the disclosure. (a) Each spot was formed by dispensing 10 drops of BSA followed by 10 drops of EDC, repeated for two layers. (b) Each spot was formed by dispensing 20 drops of BSA and 20 drops of EDC in a single layer, resulting in spot merging due to the larger volume and increased bioink spreading on the substrate. The brighter center indicates the coffee ring effect caused by uneven evaporation. (c) Each spot was formed by dispensing 20 drops of BSA and 20 drops of EDC with increased distance between spots to prevent merging. This adjustment reduced the number of microarrays. The non-uniform morphology was caused by excessive bioink evaporation in each spot and inefficient crosslinking with EDC. The scale bar is 500 μm for all the images.

FIG. 20 shows hydrogel microarrays and circularity of the microarrays in exemplary embodiments of the disclosure. (a-b) Hydrogel microarrays printed with two layers using (a) 1% BSA and (b) 2% BSA. Increased concentrations of BSA result in misalignment during printing when multiple layers are used. Scale bar is 500 μm. (c) Circularity of the microarrays printed with different concentrations of BSA and varying numbers of layers. Circularity is defined as (4π×Area)/Perimeter, where a value of 1 indicates a perfect circle. Misalignments during printing lead to lower circularities.

FIG. 21 shows images of Bioink 1 droplets formed via the Piezo Dispense Capillary (PDC) nozzle before landing on the substrate in exemplary embodiments of the disclosure. The droplet size ranges from 250 to 300 pL. (a) A 1% BSA concentration resulted in uniform droplet formation aligned with the center of the nozzle. (b) A 2% BSA concentration often led to irregular droplet formation and misalignment in micropatterning.

FIG. 22 shows application of 3D microarrays for IFA-based detection of cytokines and cytokine receptors in exemplary embodiments of the disclosure. (a) MFI obtained by IL-6 IFA in buffer at the concentration of 2500 pg/mL. The resulted fluorescence signal was significantly higher upon the application of 3D hydrogels compared to 2D microdots (P<0.0001). Error bars represent standard deviation. (b) Schematic comparison between the application of 2D microdots and 3D hydrogels in IFA. Higher surface area in 3D microarrays brought about amplified signal. (c) Fluorescence microscopy images of the 2D microdots compared to the 3D hydrogels after IL-6 IFA, in which clear enhancements in signal and consistency are seen in the 3D hydrogels. Scale bar is 200 μm. (d) Calibration curve achieved by IL-6 IFA in buffer using 3D hydrogel microarrays with an experimental LOD of 0.3 pg/mL and linearity in the range of 0.3-312.5 pg/mL (R2=0.99). (c) Calibration curve obtained by IFA of TNF R1 in buffer using 3D hydrogel microarrays with an experimental LOD of 1 pg/mL and linearity in the range of 1-2500 pg/mL (R2=0.98). (f) MFIs of IFA-based multiplex detection of both IL-6 and TNF R1 in buffer at the concentrations of 2500 pg/mL and 312.5 pg/mL, with accompanied (g) multiplex fluorescence images of 2500 pg/mL compared to control, depicting TNF R1 detection and IL-6 detection. Scale bar is 200 μm. (h) Detection of IL-6 spiked at the concentrations of 2500 pg/mL, 312.5 pg/mL, and 40 pg/mL, in blood, platelet poor plasma, and serum (P*<0.05, P**<0.01, P***<0.001, P****<0.0001). Error bars in all the plots represent standard deviation.

FIG. 23 shows MFI and representative images of the 3D hydrogels in an IL-6 IFA, in which either IL-6 or TNF R1 antigen were added separately, showing the specificity of the 3D hydrogels to IL-6 in exemplary embodiments of the disclosure. Error bars represent standard deviation (p****<0.0001). Scale bar is 200 μm.

FIG. 24 shows representative images of the IL-6 calibration curve at each IL-6 concentration tested in exemplary embodiments of the disclosure. Scale bar is 200 μm.

FIG. 25 shows MFI of IL-6 sandwich-IFA for each IL-6 concentration in exemplary embodiments of the disclosure. Error bars represent standard deviation based on T-Test Analysis (p**<0.01).

FIG. 26 shows fluorescence microscopy images of the 2D microdots and 2D projection of micro domes in exemplary embodiments of the disclosure. (a) Fluorescence microscopy images of the 2D microdots compared to the 3D hydrogels after IL-6 IFA with 2500 pg/ml IL-6 concentration. Scale bar is 200 μm (b) 2D projection of micro domes captured by an inverted fluorescence microscope.

FIG. 27 shows (a-c) Wells coated with (a) one layer of BSA gel (negative control), (b) one layer of BSA/Antibody gel (positive control), and (c) one layer of BSA/Antibody gel covered by a layer of BSA gel (BSA+BSA/Antibody), (d-f) fluorescence images of (d) one layer of BSA, (c) one layer of BSA/Antibody gel, and (f) BSA+BSA/Antibody. Scale bar is 2 mm, (g) MFI obtained from different samples in exemplary embodiments of the disclosure.

FIG. 28 shows representative images of the TNF R1 calibration curve at each TNF R1 concentration tested in exemplary embodiments of the disclosure. Scale bar is 200 μm.

FIG. 29 shows MFI of TNF R1 sandwich-IFA for each TNF R1 concentration in exemplary embodiments of the disclosure. Error bars represent standard deviation based on t-test analysis (p*<0.1).

FIG. 30 shows MFI and representative images of 3D hydrogels submerged in blood donor samples diluted in HEPES from 1×-8×, with a control of HEPES only buffer in exemplary embodiments of the disclosure. Error bars represent standard deviation (p***<0.001). Scale bar is 200 μm.

FIG. 31 shows 3D hydrogels submerged in 2× blood samples (diluted in HEPES), compared to HEPES only control in exemplary embodiments of the disclosure. Light circles indicate 3D hydrogels, while dark circles depict the presence of blood cells. Scale bars are 200 μm and 100 μm.

FIG. 32 shows the application of 3D hydrogel microarrays for IFA-based monitoring of IL-6 level in the blood of patients diagnosed with sepsis. (a) A schematic highlighting the potential clinical applications of monitoring disease progression of individuals with bacterial infections, resulting from a wide range of pathogens. Increased IL-6 levels in blood plasma can be quantified above clinical thresholds, where recovery or worsening symptoms can be monitored over time. (b) The collection of blood samples at regular intervals from sepsis patients, each with different bacterial infection, and the subsequent separation of plasma allows for precise IL-6 profiling using our 3D hydrogel microarrays to monitor patient disease progression. The 3D microarrays are directly printed in well-plates through noncontact dispensers for high-throughput applications. Elevated IL-6 induced fluorescence could be associated with upregulation of IL-6 and consequently sepsis severity, while reductions in IL-6 induced fluorescence could be correlated with recovery. (c) MFI results of IL-6 IFA in plasma samples of septic patients, using our new 3D hydrogel platform. (d) IL-6 concentrations calculated based on graph and the calibration curve in FIG. 4d (P<0.0001). Limit of linearity, as previously identified, is 312.5 pg/mL and threshold of healthy IL-6 levels is below 43.5 pg/mL.80 Error bars represent standard deviation. (c) Detection of IL-6 from the blood of a patient diagnosed with sepsis caused by S. aureus. Scale bar is 200 μm.

FIG. 33 shows representative images of IL-6 IFA in plasma samples of septic patients, using a new 3D hydrogel platform in exemplary embodiments of the disclosure. Scale bar is 200 μm.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

It will be understood that any component defined herein as being included may be explicitly excluded by way of proviso or negative limitation, such as any specific compounds or method steps, whether implicitly or explicitly defined herein. For example, in aspects, the first bioink is free of a crosslinking agent.

### II. Bioinks

Described herein is a pair of bioinks that are used in tandem to print a biosensor. The first bioink is free of a crosslinking agent, which advantageously avoids gelling in the print nozzle and reduces the chance of clogging. The first bioink comprises a scaffolding moiety, which is typically a protein, and a biorecognition element for detecting a marker in a sample. The first bioink is for printing on a substrate. The second bioink can be printed from the same or a different nozzle and comprises a crosslinking agent. The second bioink is generally printed directly on top of the first bioink and, following an optional incubation period, results in crosslinking the first bioink to form the biosensor.

Typically, the scaffolding moiety is described herein as being bovine serum albumin (BSA). It will be understood, however, that any scaffolding moiety can be used as long as it is capable of being crosslinked and can be decorated with a biorecognition element. Typical examples include BSA, collagen, gelatin, alginate, chitosan, or any combination thereof.

Further examples of scaffolding moieties include, but are not limited to polytetrafluoroethylene (PTFE), polyvinylchloride (PVC), polyethylenes (PE), polypropylenes (PP), polystyrenes, polyacrylonitrile (PAN), polymethylmethacrylate (PMMA), polyvinylacetate (PVAc), polyphenylene oxide, polypropylene oxide (PPO), polyvinylidene fluoride (PVDF), polybutylene, polyamides (PA, Nylons), polyesters, polycarbonates, polyurethanes, polysiloxanes, polyimides, polyetheretherketone (PEEK), polysulfones, polyethersulphone, cellulose, polysaccharides and their derivatives, acrylates, methacrylates (eg. methyl methacrylate), ethylene, propylene, tetra-fluoroethylene, styrene, vinyl chloride, vinylidene chloride, vinyl acetate, acrylonitrile, 2,2-bis[4-(2-hydroxy-3-methacryloyloxy-propyloxy)-phenyl]propane (Bis-GMA), ethyleneglycol dimethacrylate (EGDMA), tri-ethyleneglycol dimethacrylate (TEGDMA), bis(2-methacrylyl-oxyethyl) ester of isophthalic acid (MEI), bis(2-meth-acryloxyethyl) ester of terephthalic acid (MET), bis(2-methacryloxyethyl) ester of phthalic acid (MEP), 2,2-bis(4-methacryloxy phenyl) propane (BisMA), 2,2-bis[4-(2-methacrylyloxyethoxy)phenyl]propane (BisEMA), 2,2-bis[4-(3-methacryloyloxy-propoxy)phenyl]propane (BisPMA), hexafluoro-1,5-pentanediol dimethacrylate (HFPDMA), bis-(2-methacrylyloxyethoxy-hexafluoro-2-propyl)benzene[Bis(MEHFP)N], 1,6-bis(methacryloyloxy-2-ethoxycarbonylamino)-2,4,4-tri-methylhexan (UEDMA), spiro orthocarbonates, other vinyl monomers, the derivatives of these monomers, diacids and diols (pairs), w-hydroxy carboxylic acids, lactones, diacids and diamines (pairs), amino acids, lactams, diisocyanates, diols (pairs), poly(lactide-co-glycolide) (PLGA), poly(L-lactic acid) (PLLA), poly(D,L-lactic acid) (PDLLA), polyglycolic acid (PGA), polyanhydrides, poly(ortho ethers), poly(ε-caprolactone) (PCL), poly(hydroxy butyrate) (PHB), poly(propylene fumarate) (PPF), polyphosphoesters (PPE), polyphosphazenes, collagen, gelatin and many other proteins, carbohydrates, and/or their derivatives, polyvinyl alcohol, polyethylene oxide (polyethylene glycol), polymethacrylic acid (PMAA), polyvinyl pyrrolidone, polyacrylic acid, poly(lysine), poly(allylamine), poly(ethylenimine), poly(acrylamide), poly(acrylamide-co-acrylic acid), poly(acrylamide-co-diallyldimethylammonium chloride), polyethylene-block-poly(ethylene glycol), poly(propylene glycol), poly(2-hydroxypropyl methacrylate), poly(2-hydroxyethyl methacrylate), poly(4-hydroxystyrene), polyethylene monoalcohol, poly(vinyl alcohol-co-ethylene), poly(styrene-co-allyl alcohol), hydroxyethylcellulose, alginate, pectin, chitin, chitosan, dextran, hyaluronic acid, collagen, gelatin, acrylic acid, methacrylic acid, 4-vinylbenzoic acid, crotonic acid, oleic acid, elaidic acid, itaconic acid, maleic acid, fumaric acid, acetylenedicarboxylic acid, tricarballylic acid, sorbic acid, linoleic acid, linolenic acid, eicosapentanoic acid, other unsaturated carboxylic acids, anhydrides, their derivatives, other organic acids such as sulfonic acid, and/or phosphonic acid replacement of the carboxyl group of the above listed unsaturated carboxylic acids, their derivatives, allylamine, 4-vinylaniline, L-lysine, D-lysine, DL-lysine, acrylamide, their derivatives, 2-hydroxypropyl methacrylate, 2-hydroxyethyl methylacrylate, 4-hydroxystyrene, ethylene glycol, propylene glycol, poly(ethylene glycol) acrylate, poly(ethylene glycol) methacrylate, their derivatives, and/or any mixtures thereof.

The scaffolding moiety may be present in the first bioink in any amount to achieve a functional biosensor. This can be adjusted based on the scaffolding moiety chosen, the biorecognition element chosen, and the crosslinking agent used. To determine an appropriate amount, typically it is desired that the resulting printed biosensor has a consistent morphology and circularity.

For example, the scaffolding moiety may be present in an amount of from about 0.1% to about 20% by weight of the first bioink, such as from about 0.1%, 0.2%, 0.3%, 0.4%, 0.5%, 0.6%, 0.7%, 0.8%, 0.9%, 1%, 1.25%, 1.5%, 1.75%, 2%, 2.5%, 3%, 5%, 7.5%, 10%, or 15% to about 0.2%, 0.3%, 0.4%, 0.5%, 0.6%, 0.7%, 0.8%, 0.9%, 1%, 1.25%, 1.5%, 1.75%, 2%, 2.5%, 3%, 5%, 7.5%, 10%, 15%, or 20%. Typically, the scaffolding moiety is present in an amount of less than about 2% by weight of the first bioink.

Turning now to the biorecognition element will be understood to be any component that is selective for the desired target. The biorecognition element can be, for example, a receptor or a probe molecule. Typically, the biorecognition element comprises an antibody or fragment thereof, a peptide, a polynucleotide, an aptamer, an aptamer-conjugated nanoparticle, a DNAzyme, a bacteriophage, or any combination thereof.

Similarly, the crosslinking agent can be any agent capable of crosslinking the scaffolding moiety. This may vary depending on the scaffolding moiety chosen. Typically, the crosslinking agent comprises 1-ethyl-3-(3-dimethylaminopropyl) carbodiimide (EDC), N-hydroxysuccinimide (NHS), glutaraldehyde, N′,N′-dicyclohexyl carbodiimide (DCC), N,N′-diisopropyl carbodiimide (DIC), sulfo-NHS, thiophosgene, succinic anhydride, 3-mercaptopropyl trimethoxysilane (MPTMS), Y-maleimidobutyryloxy succinimide (GMBS), or any combination thereof. The crosslinking agent may be present in any amount sufficient to crosslink the first bioink. In typical aspects, this amount is sufficient for the printed and crosslinked biosensor to withstanding intense washing with water and/or assay buffer, as described herein. For example, the crosslinking agent is typically present in an amount of about 0.1 mg/ml to about 20 mg/ml, such as from about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 1.25 mg/ml, 1.5 mg/ml, 1.75 mg/ml, 2 mg/ml, 2.5 mg/ml, 3 mg/ml, 5 mg/ml, 7.5 mg/ml, 10 mg/ml, or 15 mg/ml to about 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 1.25 mg/ml, 1.5 mg/ml, 1.75 mg/ml, 2 mg/ml, 2.5 mg/ml, 3 mg/ml, 5 mg/ml, 7.5 mg/ml, 10 mg/ml, 15 mg/ml, or 20 mg/ml. Typically, the crosslinking agent is present in an amount of about 2 mg/mL.

The substrate upon which the bioinks are printed may be anything upon which the bioinks will bond. Typically, the substrate comprises glass, a thermoplastic substrate, cyclic olefin copolymer, polystyrene, polycarbonate, thermoset, polydimethylsiloxane, fabric, paper, cellulose, or any combination thereof. The substrate may be modified to improve bonding of the bioinks. For example, when the substrate is glass, it is typically fluorosilanized to yield surface hydrophobicity while preserving some functional carboxyl groups on the surface.

Also described herein is a biosensor. The biosensor is made from the pair of bioinks described herein. The first bioink is printed on the substrate and then the second bioink is printed on top of the first bioink. In this way, crosslinking only occurs on the substrate rather than in the print nozzle.

The pair of bioinks may be layered to produce the biosensor. Typically, a first layer of bioinks is applied followed by at least a second layer. Further third or fourth or so on layers may be applied as desired. Typically the second bioink is applied directly on top of the first bioink without any intervening layers. Similarly, the second layer of the first bioink is then applied directly on top of the first layer of the second bioink without any intervening layers.

A microarray is provided herein, where a plurality of the biosensors are applied to a substrate. It is contemplated that the microarray may be capable of detecting more than one marker by applying biosensors to the substrate with different biorecognition elements. In this way, two or more markers can be detected using a single microarray.

Also described herein is a method of printing a biosensor from the pair of bioinks described herein. The method comprises printing the first bioink onto the substrate; and subsequently printing the second bioink onto the first bioink. As described above, this process may be repeated one or more times to produce multiple layers of the bioinks. For example, the method typically further comprises printing a second layer of the first bioink onto the second bioink followed by printing the second bioink onto the second layer of the first bioink.

It is contemplated that there may be an incubation period between printing the first layers of inks and any second or third or more layers so that the first layer has sufficient time to crosslink before a subsequent layer is applied.

Printers used for printing bioinks may have one or more than one nozzle. It is contemplated that the first and second bioinks may be printed from the same nozzle in order or that the first bioink is printed from a first nozzle and the second bioink is printed from a second nozzle.

### Examples

The following non-limiting examples are illustrative of the present disclosure:

### Results and Discussion

Surface Treatment and Two-Step Bioprinting for 3D Biosensing Microarray Fabrication: To fabricate the 3D biosensing platform, glass substrates were first activated by carbon dioxide (CO2) plasma treatment and fluorosilanized through chemical vapor deposition (CVD) of trichloro(1H, 1H,2H,2H-perfluorooctyl) silane (TPFS) (4,47-49). An optimized fluorosilanization technique was developed by adjusting the CVD time to 30 min and the subsequent heat treatment to 15 min. This approach allows for only partial consumption of the carboxyl groups induced by plasma treatment through reaction with fluorosilane (FS) groups. This results in surface hydrophobicity while preserving some functional carboxyl groups on the surface.

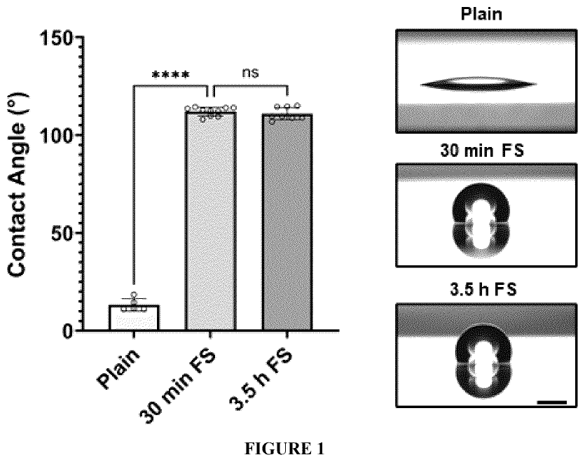

The degree of hydrophobicity induced by the deposited FS groups prevented the printed droplets from spreading upon contact with the substrate, enabling the formation and retention of a 3D shape. Additionally, the larger volume of each 3D-shaped hydrogel retained moisture for a longer period, reducing evaporation rates compared to 2D spread droplets (41) This minimized drying of the droplets throughout the printing stages. As shown in FIG. 1, the water contact angle after the optimized protocol with 30-minute CVD treatment reached 111°, which is comparable to the contact angles obtained after conventional FS CVD treatment with a longer incubation (50-52).

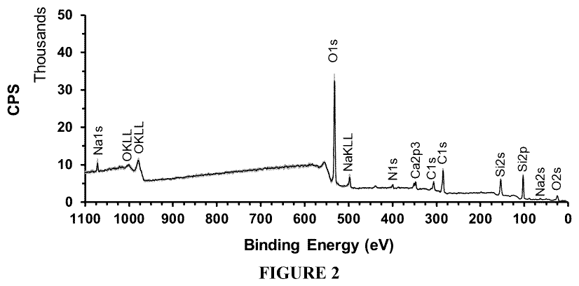

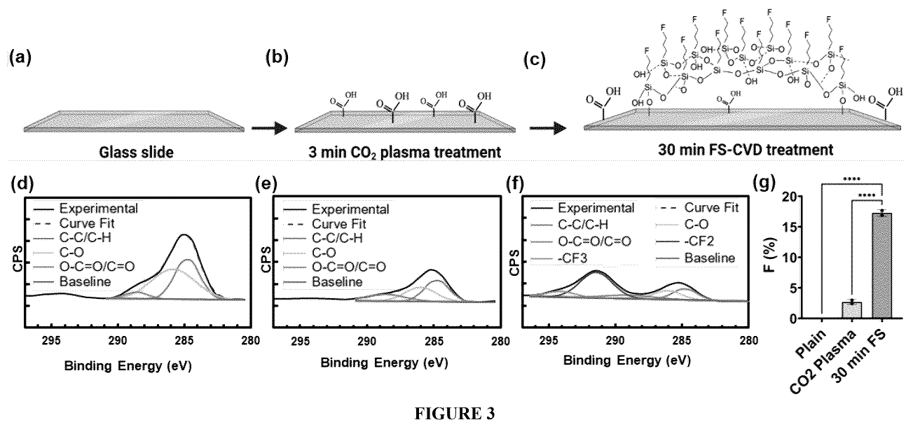

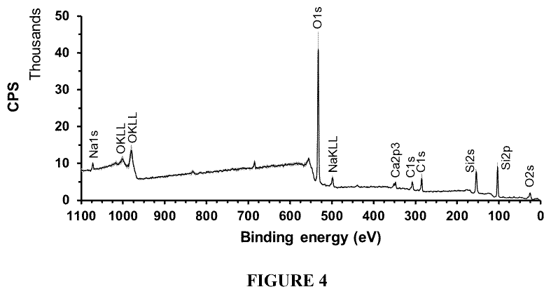

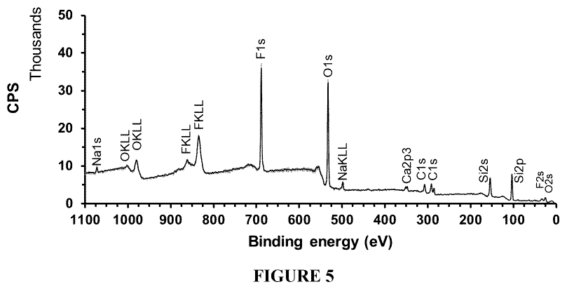

The remaining carboxyl groups after FS CVD treatment are utilized for covalent attachment of the hydrogel microarrays to the hydrophobic substrate after printing (4,53). To characterize and determine the chemical structures present on the treated substrates, X-ray photoelectron spectroscopy (XPS) was conducted during each step of the functionalization process, including before treatment (FIG. 2 and FIG. 3a), after 3-minute CO2 plasma treatment (FIG. 4 and FIG. 3b), and after 30-minute CVD with TPFS (FIG. 5 and FIG. 3c) (4). Deconvolution plots for each treatment step were then constructed through Gaussian distribution of the C1s spectra peak (FIG. 3d-f). From the XPS results, atomic percent concentration of each element (Table 1) as well as peak area percentages (Table 2) of each chemical bond were quantified to determine changes in chemical composition and binding after each treatment step.

TABLE 1
Atomic concentration percentage values and standard deviations
for each element found on the plain untreated substrate, CO₂ plasma-treated
substrate and 30-min fluorosilanized substrate

| | Atomic Concentrations (%) | | | | | | |
|---|---|---|---|---|---|---|---|
| | C | O | Na | Si | Ca | N | F |
| Plain | 23.4 | 3.25 | 53.72 | 3.43 | 14.35 | 1.83 | 0 |
| | (±1.47) | (±1.4) | (±0.59) | (±0.42) | (±0.08) | (±0.01) | |
| CO₂ | 9.11 | 66 | 2.07 | 18.51 | 1.6 | 0 | 2.71 |
| plasma | (±1.46) | (±2.01) | (±0) | (±0.01) | (±0.15) | | (±0.39) |
| treated | | | | | | | |
| 30 min | 13.95 | 50.46 | 2.39 | 14.51 | 1.41 | 0 | 17.27 |
| FS | (±1.24) | (±1.25) | (±0.01) | (±0.46) | (±0.11) | | (±0.55) |

TABLE 2
Peak area percentages calculated through deconvolution of C1s for each step of substrate
treatment (errors represent standard deviation)

| | Peak Area (%) | | | | |
|---|---|---|---|---|---|
| | C—C/C—H | C—O | O—C═O/C═O | —CF2 | —CF3 |
| Plain | 42.4 (±2.34) | 48.95 (±5.42) | 8.64 (±3.07) | 0 | 0 |
| CO2 Plasma Treated | 43.07 (±0.11) | 36.86 (±0.26) | 20.06 (±0.37) | 0 | 0 |
| 30 min FS | 16 (±0.87) | 13.57 (±1.09) | 11.13 (±1.87) | 50.52 (±2.4) | 8.77 (±2.3) |

Untreated glass slides showed the highest carbon percentage (23.40%) and lowest oxygen percentage (3.25%) among the three treatment steps. Nitrogen and sodium impurities were also present on the untreated substrate, which were etched away during the plasma-treatment. Similarly, the C1s spectra peak deconvolution showed that C—C/C—H (42.40%) and C—O (48.95%) were most presented on the untreated substrate, along with little indication of carboxyl groups (8.64%). After CO2 plasma-treatment, the peak at the O1s region increased and oxygen percentage reached 66.00% while carbon concentration dropped to 9.11%. Considering the peak area percentages at C1s, the O—C—O/C═O chemical bonds displayed a larger peak (20.06%), and a subsequent drop in C—O (36.86%). These results show that methoxy groups that were originally abundant on untreated substrates, were largely replaced by carboxyl groups through CO2 plasma-treatment. Although a slight trace of fluorine (2.71) could be found in the chemical structure of plasma-treated substrates, no CF2/CF3 bonds were observed at C1s deconvoluted peaks.

After CVD treatment of the substrate with TPFS, the atomic concentration of fluorine drastically increased to 17.27%. As seen in FIG. 3g, FS treatment led to significantly higher amount of fluorine in the XPS results, compared to plasma-treated and plain slides. The CF2 functional group became the predominant chemical bond, reaching peak area percentage of 50.52%, accompanied by CF3 bonds, at 8.77%. Furthermore, the appearance of O—C═O/C═O (11.13%) in C1s deconvoluted peaks after FS treatment confirm the functionality of the surface for covalent immobilization of biomolecules via carboxyl-amine binding.

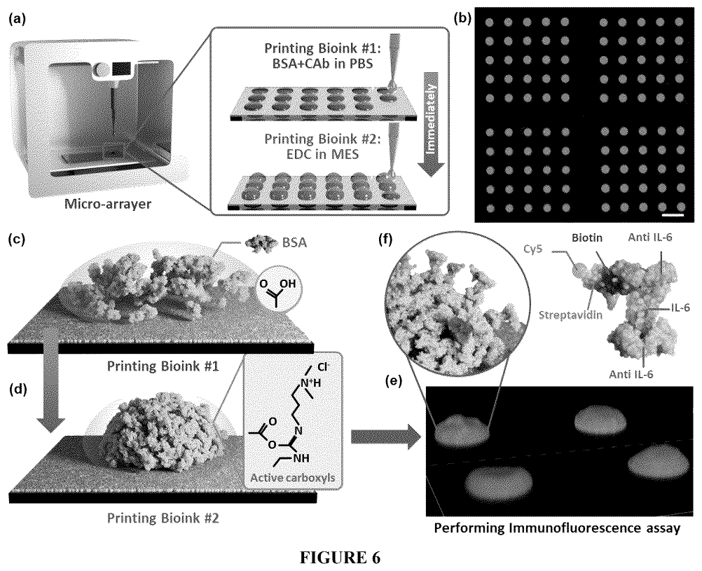

Before printing, the printer's chamber was set to a relative humidity of 65% and a stage temperature of 18° C. to maintain a constant dew point, balancing evaporation and condensation to prevent droplet drying (54,55). Printing was then performed on the FS-treated substrates using an automated non-contact, two-step bioprinting approach (FIG. 6a).

The preliminary step involved printing of BSA-based bioink (bioink #1) containing 1-2% BSA in phosphate-buffered saline (PBS) and 150 μg/mL of either IL-6 or tumor necrosis factor receptor 1 (TNF R1) CAbs. TNF R1 was chosen rather than TNF cytokine for multiplex detection, as it is more stable and reliable for clinical diagnostics and can be found free floating in blood (42-46). Afterwards, a secondary bioink (bioink #2) consist of 1-2 mg 1-Ethyl-3-(3Dimethylaminopropyl) carbodiimide (EDC) per 1 mL 2-(N-morpholino) ethanesulfonic acid (MES) was dispensed directly above the preliminary BSA printed dots. The printing pattern comprised 5×5 microarray blocks, in a way that each block could fit into a separate well upon the placement of a superstructure on the printed substrate (FIG. 6b). Gelation of the hydrogel occurred only after the EDC cross-linker was printed onto the substrate, rather than inside the nozzle. This approach significantly reduces the risk of nozzle blockage, a common issue in non-contact printing methods where premature gelation can occur within the nozzle (FIG. 6c-d) (56-58).

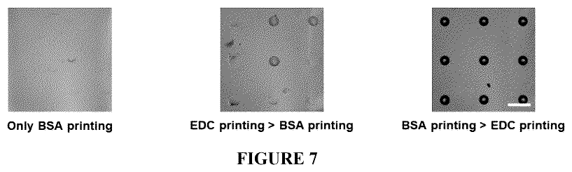

EDC plays two roles in the formation of these 3D hydrogels. Firstly, it crosslinks the carboxyl groups of both BSA and CAbs to the amine groups of adjacent proteins and antibodies, enabling the formation of a dense protein network. Secondly, it crosslinks carboxyl groups present on the CO2 plasma-activated substrate with amine groups present on the crosslinked protein network. These two mechanistic roles mean that printing order is critical, as validated in FIG. 7, which established that the 3D hydrogels were not stable if EDC was printed before BSA. This is because EDC fully reacts with the carboxyl-rich glass substate in the absence of BSA and CAbs, inhibiting effective protein crosslinking when proteins are subsequently deposited.

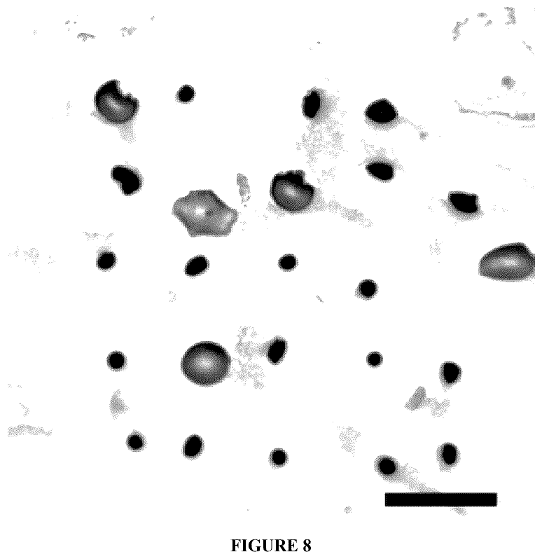

Printing EDC first at high concentrations also led to inconsistency in spot formation. This could be due to the high viscosity of the print solution which affects the nozzle's ability to generate stable droplets. For instance, at 3 mg/ml EDC, the lack of stability and repeatability in droplet formation significantly disturbed printing precision. As a result, missing spots and irregular patterns formed after printing, as illustrated in FIG. 8. Another major problem with high EDC concentrations is the formation of a large amount of salt on the substrate. Although the environment and dew point of the printing chamber are controlled, slight evaporation of the printed spots is expected due to the low volumes of print (typically in the 2-3 nano liters range). When the EDC concentration is increased, it leaves a very thick layer of salt on the substrate. This remaining salt layer alters the hydrophobicity of the substrate, causing the second spotted bioink to not align precisely on the same spots. Additionally, the volume of the second spotting is not sufficient to dissolve the salt layer quickly after printing.

For each print, 10 droplets of bioink were dispensed per spot, with each droplet having a volume of 250-300 pL. The protocol begins by printing 10 droplets per spot of BSA (bioink #1), followed by 10 droplets per spot of EDC (bioink #2) to create the hydrogel patterns. After a 30-minute incubation, additional cycles of BSA/EDC prints were performed. This introduced multiple layers of protein networks, which increased the bio-functional volume of the 3D hydrogels and improved their uniformity (FIG. 6c). The substrate was incubated overnight in 100% humidity-controlled conditions at 4° C., to prevent any potential hydrogel drying after printing, and then used in a sandwich-based IFA (FIG. 6f).

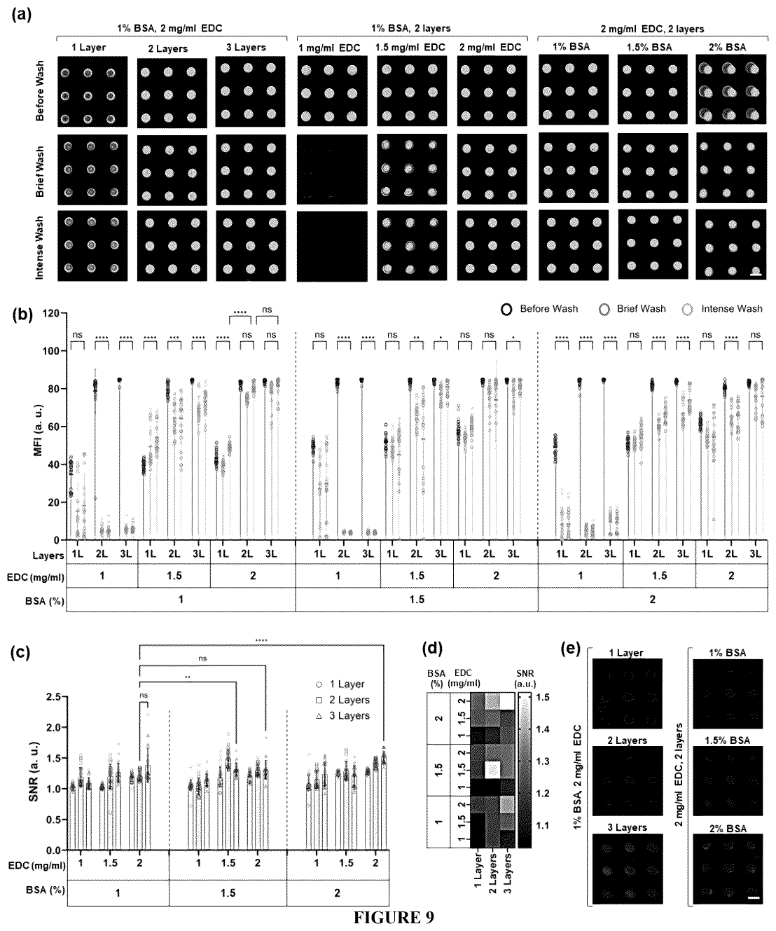

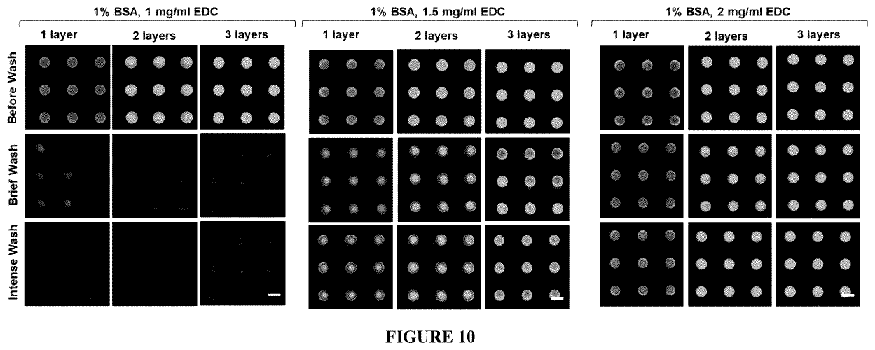

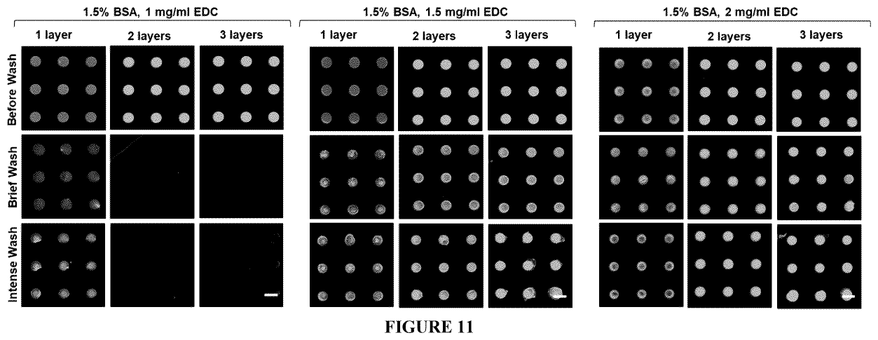

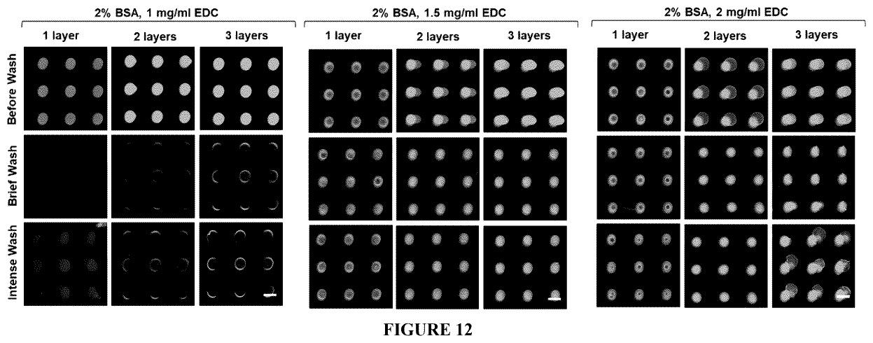

Optimization of 3D Proteinaceous Microarrays: The concentration of BSA, EDC, and number of layers was optimized in this 3D printing fabrication process, whereby hydrogel stability and non-specific attachment were analyzed. To assess the stability of the 3D hydrogels, fluorescently labeled BSA-fluorescein isothiocyanate (BSA-FITC) was substituted with CAbs at an equal concentration. The 3D hydrogels were first briefly washed, followed by an intense wash. Fluorescence intensity images depicted the visual changes for each washing stage (FIG. 9a, FIG. 10, FIG. 11, and FIG. 12).

Increasing BSA concentrations from 1% to 2% did not show any visible trend in the fluorescence intensities of different samples (FIG. 9b). The BSA concentration did not significantly affect the stability of the hydrogel microarrays. The lack of observed difference in fluorescence intensity with increasing BSA concentration may be due to the limitations of the microscope and potential over-saturation of the fluorescence signal. However, it was later demonstrated, increasing the BSA concentration to 2% leads to a decrease in the consistency of the microarray morphology and circularity. Therefore, 1% BSA was identified as the most reliable for the patterning application.

On the other hand, EDC plays a key role in hydrogel stability as it enables effective crosslinking. The lowest concentration of EDC, at 1 mg/mL, exhibited an almost complete loss of fluorescence upon brief washing (e.g., 2 L-1% BSA-1 mg/mL EDC in FIG. 9a), which can be attributed to insufficient crosslinking. Here, the low EDC concentration not only had poor hydrogel gelation but were unable to withstand the shear stresses of the subsequent sandwich IFA. When EDC concentration increased to 1.5 mg/mL, fluorescence intensity was partially retained after brief washing (e.g., 2 L-1% BSA-1.5 mg/mL EDC in FIG. 9a). However, there was still a significant reduction in mean fluorescence intensity (MFI) after an intense wash (FIG. 9b). Additionally, EDC enhances the hydrogel's hygroscopic properties, via the deposition of active carboxyl groups on the hydrogel's surface. Carboxyl functional groups, due to their high polarity and chemical structure, allow for increased moisture absorption and retention within each hydrogel in the controlled humidity environment of the non-contact printer's incubation chamber. As EDC concentrations increased, the increase of hygroscopic properties caused by higher levels of active carboxyl groups reduced incidences of droplet evaporation, preventing droplet drying and thus, contributing to the hydrogel's overall stability (55,59,60). The highest concentration of EDC, at 2 mg/mL, maintained the foremost integrity of the 3D hydrogels by maximizing crosslinking while also increasing hygroscopic properties, resulting in a small reduction in MFI after an intense wash. Printing EDC concentrations greater than 2 mg/mL was also attempted, however, the high reactivity, salt formation and viscosity of the bioink resulted in extremely high nozzle blockage, and printing was not possible.

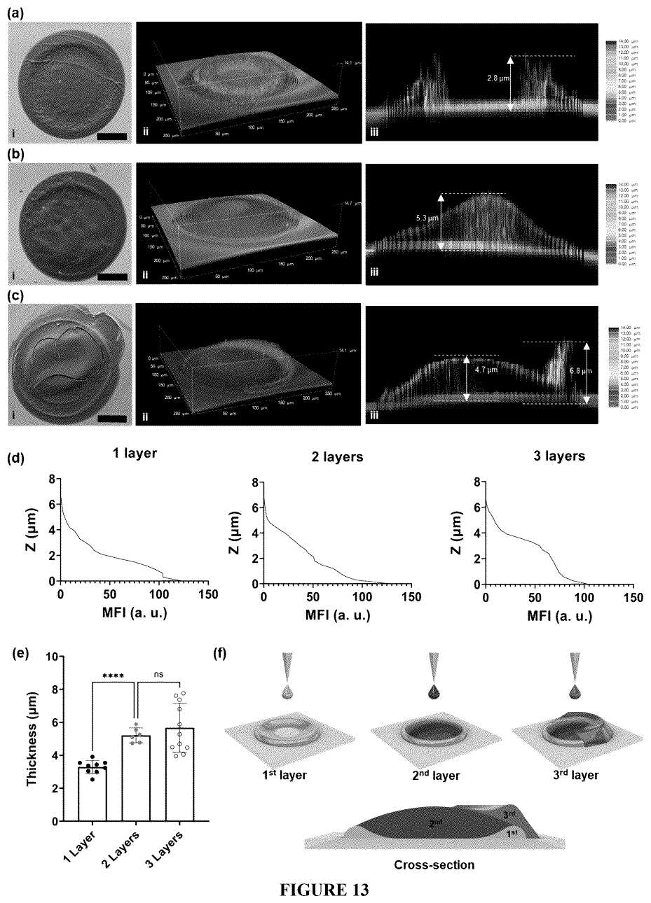

With regards to the number of print layers, it was evident that 1-layer hydrogels had the lowest intensities amongst all samples. This was expected due to the lower amount of printed BSA-FITC. Single-layer hydrogels also depicted a coffee ring effect that were not present in hydrogels with 2 or 3 layers, as is evident in IL-1% BSA-2 mg/mL EDC fluorescent images shown in FIG. 9a and FIG. 13. As reported in literature, the coffee ring effect is a result of the tendency of fluid flow within sessile droplets to move to the boundary of the droplet, with the proteins concentrating at the boundary due to surface tension (39,61,62). This phenomenon can often be controlled by the introduction of drying processes, temperature control, and humidity control (62). However, such strategies did not yield any improvement in the observable coffee-ring effects of 1-layer hydrogels. On the other hand, the drying steps introduced during 2-layer and 3-layer hydrogel fabrication eliminated coffee-ring formation, yielding uniform semi-sphere microdots. All three-layer conditions exhibited strong fluorescence retention, which confirmed strong structural integrity. Furthermore, this validated the covalent attachment between the stacked layers of the 2-layer and 3-layer hydrogels. In general, 3D hydrogels with 2 mg/mL EDC that had 2-layers or 3-layers possessed the best shape integrity, consistency, stability, and MFI among all samples.

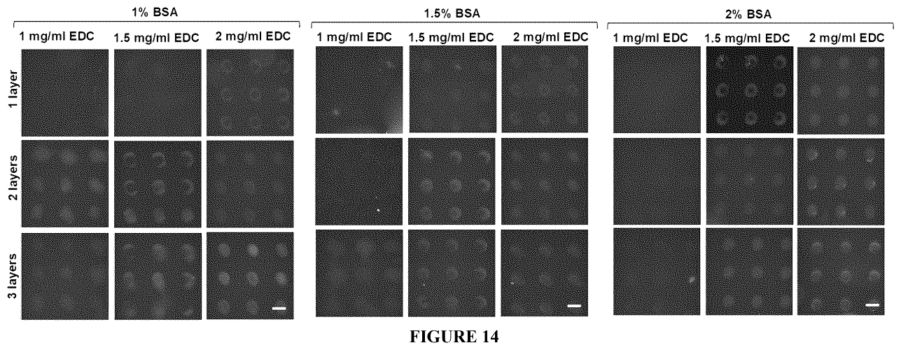

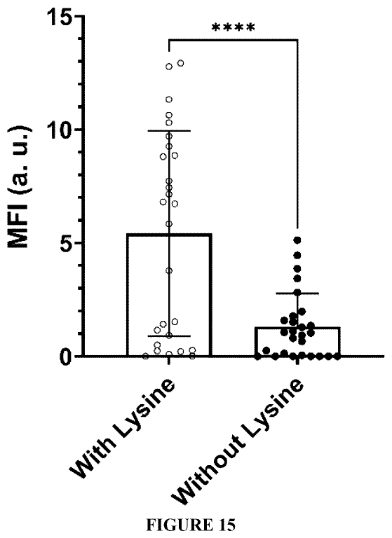

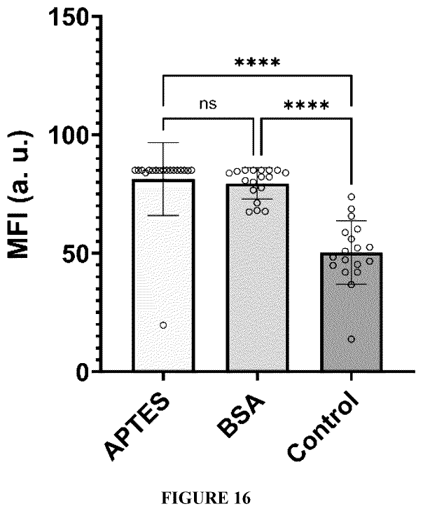

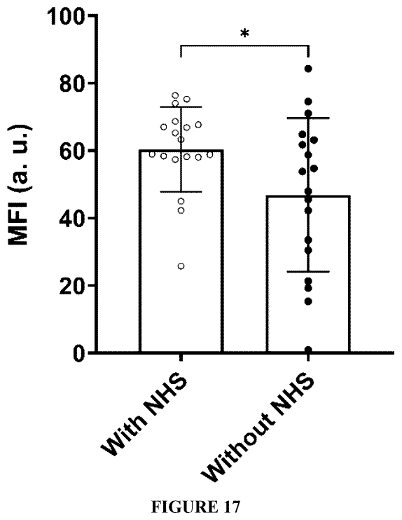

The susceptibility of these 3D hydrogels to non-specific biomolecule attachment was assessed, which would yield high background noise and diminished specificity within biosensing applications (FIG. 9c-d, FIG. 14, FIG. 15, FIG. 16, FIG. 17). All variations of the 3D hydrogels were printed with IL-6-specific CAbs. The microarrays were then directly incubated with streptavidin-Cy5, without the IL-6 biomarker and secondary detection antibodies (DAbs). A hydrogel with high specificity to IL-6 would not bind to streptavidin-Cy5 in the absence of IL-6 biomarker and DAbs. As such, non-specific attachment would be observable through high background noise and fluorescent signals detected from the hydrogel microarrays. The signal-to-noise ratios (SNRs) resulting from the non-specific attachment of the 3D hydrogels directly with Cy5 are plotted in FIG. 9c-d and representative fluorescence images are demonstrated in FIG. 9c. From the findings, any noticeable change in SNR when increasing the BSA concentration was not observed. On the other hand, increased EDC concentration and hydrogel thickness-through the addition of a third layer, increased non-specific SNR values (e.g., 3 L-1% BSA-2 mg/mL EDC hydrogels in FIG. 9c).

The background noise in the hydrogel microarrays could result from the non-specific attachment of biomolecules to the remaining amine groups and EDC-activated carboxyl groups after hydrogel crosslinking. Commonly used reagents, such as lysine (FIG. 15), (3-aminopropyl)triethoxysilane (APTES), and additional BSA were tested (FIG. 16), to neutralize the remaining activated carboxyl groups and suppress non-specific attachment. However, in all conditions, the background noise increased, likely due to the high concentration of positively charged amine groups introduced by these reagents, which results in electrostatic adsorption of biomolecules. N-hydroxysuccinimide (NHS) was also tested during the printing process to improve hydrogel formation (FIG. 17). However, NHS caused higher background noise, potentially due to its effect on reducing the crosslinking rate of the hydrogels and leaving additional non-reacted NHS esters within the hydrogel matrix (63,64).

The background noise was minimized by fine-tuning the concentrations of EDC and BSA, as well as adjusting the number of printed layers in the system, achieving a balance between carboxyl and amine groups (65). This approach, combined with the inherent blocking capability of BSA, allowed us to prevent non-specific attachment without the need for additional blocking agents, thereby simplifying the IFA process (66-68).

Scanning electron microscopy (SEM) was utilized to show surface topography of the proteinaceous hydrogels. Moreover, to determine the geometry and thickness of the 3D microstructures, confocal microscopy imaging was performed while the 3D hydrogels were submerged in PBS to prevent hydrogel cracking because of dehydration (FIG. 13a-c). FIG. 13ai demonstrates the formation of 1-layer hydrogel donut-shapes due to the coffee ring effect, which was further confirmed by the orthogonal projection of the 3D structure from confocal microscopy (FIGS. 13aii and 13aiii). Measuring the depth of the 3D structure showed a thickness value of Oum in the center of the donut shape with an outer thickness of ˜2.8 μm. In the 2-layer hydrogels (FIG. 13b), it appears that the donut shape was filled with the introduction of the second layer by increasing volume, resulting in a smooth surface topography and homogenous thickness. Also, the confocal microscopy orthogonal projection showed a smooth curvature in the 3D hydrogel structure and a thickness of ˜5.3 μm at the peak. Addition of a third layer resulted in excess hydrogel contents pooling or accumulating at the outermost edges. This was evident in the SEM images (FIG. 13ci), where the edges of the surface topography showed leaking of the excess hydrogel contents around the edges and an uneven circular border. The confocal microscopy further illustrates the pooling effect towards the outermost border, resulting in a thickness of ˜6.8 μm at the border, while the central thickness resembles the thickness of the 2-layer hydrogel, at ˜4.7 μm (FIGS. 13cii and 13ciii).

Analysis of the MFI along the z direction demonstrated the distribution of FITC, as a placeholder for CAbs, throughout the hydrogel thickness. As shown in FIG. 13d, the 1-layer hydrogel had the highest MFI values at the base of the hydrogel microstructure, which sharply dropped as thickness increased. This corroborated the donut-shape of the hydrogel, whereby the gap in the center of the structure led to a nonuniform distribution of hydrogel content. In the 2-layer and 3-layer hydrogels, FITC showed better distribution across the thickness with the most linearity at the 2-layer, demonstrating the uniform distribution of hydrogel content. FIG. 13e exhibits the average thickness of the printed 3D hydrogels versus the number of layers. Measurements of the edge thickness and middle thickness were taken for each microarray hydrogel spot, which were then averaged between all hydrogel spots of the microarray. This calculation was done separately for each hydrogel microarray to obtain the thicknesses for 1-layer, 2-layer, and 3-layer hydrogels. There was no significant difference in the calculated average thickness of the 2-layer and 3-layer conditions, while the 1-layer showed significantly lower height. This suggests that pooling of the 3-layer hydrogel content towards the border was not specific to one edge of the 3D hydrogels, but rather, was random each time.

Based on the results, it was concluded that the second layer of the hydrogel fills the middle space induced due to the coffee ring effect of the first layer, creating a nicely formed dome (FIG. 13f). However, printing an additional layer causes material to accumulate at the periphery. Pooling of the 3-layer hydrogel content towards the border was not specific to one edge of the 3D hydrogels, but rather random each time.

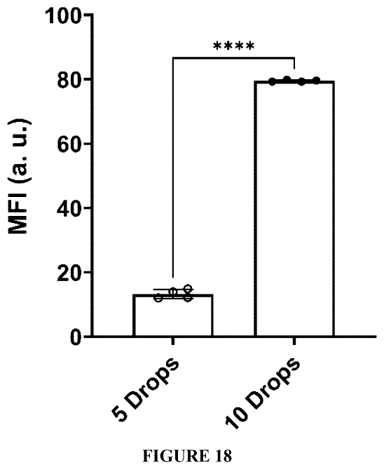

It should be pointed out that the volume of the printed bioink was also optimized in this study. This volume is determined by the number of drops that the noncontact dispenser generates at the printing spot. For optimization of the volume, a biotinylated antibody was incorporated in the proteinaceous hydrogels and incubated the hydrogel arrays with streptavidin-Cy5 to measure the resulting signal. Printing 10 drops per spot (˜3 nL) for each step of the print provides the best consistency (FIG. 18).

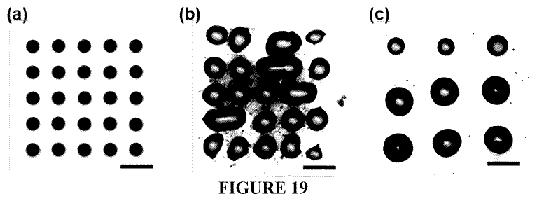

Printing larger volumes of bioinks in a single layer to printing multiple layers was compared and found several significant issues with the former approach. In a 2-layer print, a total of 20 droplets per spot are printed for each bioink, with 10 droplets applied in each layer (FIG. 19a). Printing all 20 droplets at once caused the spots to spread too far, leading to merging and reduced pattern consistency (FIG. 19b). To prevent merging, the spots needed to be placed further apart, which required a redesign and made it impossible to fit enough spots in small areas, such as a 96-well plate, for multiplex detection (FIG. 19c). Additionally, the morphology of the hydrogel microarrays was suboptimal with 20 droplets per spot, exhibiting a more pronounced coffee ring effect and failing to form the desired elevated domes that enhance sensitivity (FIG. 19b). Technological challenges with increased evaporation due to extended printing time were faced, which reduced the efficiency of EDC crosslinking and compromised spot robustness during immunoassay washing (FIG. 19c).

Ultimately, the optimized parameters for the proteinaceous hydrogels in the sandwich-IFA application were chosen to be 2 L-1% BSA-2 mg/mL EDC hydrogels (see FIG. 9c). Importantly, while the 2 mg/mL EDC yields a slightly higher non-specific SNR than lower EDC concentrations, this concentration is necessary for hydrogel stability, as discussed earlier.

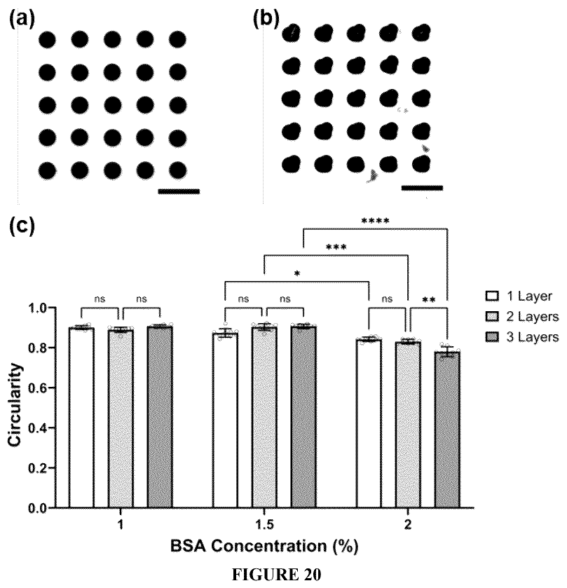

To quantitatively assess spotting alignment precision, the spot morphology in one-layer, two-layer, and three-layer prints were compared. If the printing of the second or third layers is misaligned, the spots may appear oval or irregular in shape rather than circular (FIG. 20a-b). To evaluate this, circularity of each spot was calculated. Circularity is defined as (4π×Area)/Perimeter2, where a value of 1.0 indicates a perfect circle. These circularity values were compared across different numbers of layers to assess consistency. To ensure accurate quantification, images were captured before any washing steps. This approach highlights the precision of the printer in achieving consistent printing.

The results of these experiments are shown in FIG. 20c, with the mean values, standard deviation (SD), and coefficient of variation (CV) for each condition detailed in Table 3. Circularities were close to 1 for 1% and 1.5% BSA concentrations, with no significant differences across the samples. This indicates that the second and subsequent rounds of spotting fully covered the initial spots with minimal positional deviation.

TABLE 3
Mean, standard deviation (SD), and coefficient of variation (CV) values
for the circularity of hydrogel microarrays (n = 9). Microarrays were
printed with varying numbers of layers and BSA concentrations, while
the EDC concentration was maintained at 2 mg/mL for all conditions

| BSA | No. of | | | |
|---|---|---|---|---|
| Conc. | Layers | Mean Circularity¹ | SD | CV |
| 1% | 1 | 0.900 | 0.010 | 1.11% |
| | 2 | 0.889 | 0.013 | 1.51% |
| | 3 | 0.906 | 0.008 | 0.90% |
| | 1 | 0.874 | 0.021 | 2.43% |
| 1.5% | 2 | 0.903 | 0.017 | 1.87% |
| | 3 | 0.907 | 0.011 | 1.18% |
| | 1 | 0.842 | 0.011 | 1.32% |
| 2% | 2 | 0.830 | 0.012 | 1.40% |
| | 3 | 0.780 | 0.025 | 3.17% |

${}^{1} Circularity = \frac{4 π \times Area}{{Perimeter}^{2}} ; a value of 1 indicates a perfect circle$

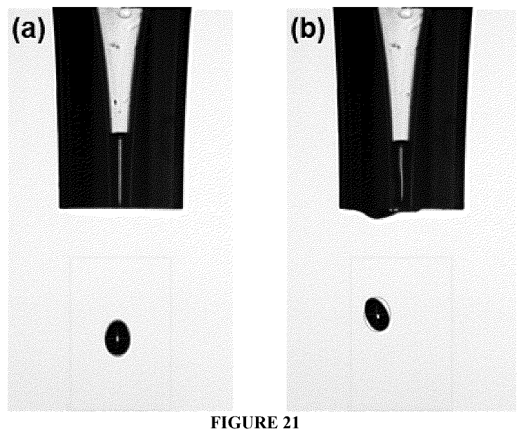

The inconsistency and misalignment in droplet formation is more problematic when the viscosity of the bioink is relatively high, preventing the droplets formed stably in the center of the nozzle and landed accurately on the same spot with each print (FIG. 21). Increasing the BSA concentration to 2% resulted in a significant reduction in circularity from 0.9 to 0.8, likely due to inconsistencies in droplet formation caused by the higher viscosity of the bioink at 2% BSA. All conditions demonstrated a CV value of less than 5%. With the optimal parameters (2 layers, 1% BSA, 2 mg/mL EDC), a CV value of 1.51% was achieved, indicating high consistency in spot formation. Additionally, 3% BSA was attempted, but yielded no stable hydrogels due to the high degree of nozzle blockage, difficulty printing and reduced robustness.

To ensure thorough characterization of the repeatability of the spot morphology, the diameter, thickness, perimeter, circularity, and solidity of the spots was measured by analyzing the hydrogel microarrays after an intense wash. These results, along with the associated standard deviations and coefficients of variation, are presented in Table 4. CV values of less than 1.2% were obtained for the repeatability of the spot morphology, indicating excellent consistency in printing across various samples. Furthermore, the CV value for the hydrogel's thickness is 8.65%, confirming that the printing process is adequately reliable for creating uniform spots.

TABLE 4
Assessment of hydrogel microarrays (n = 9) printed with 2 layers, 1% BSA, and 2 mg/ml
EDC. The table includes mean values, standard deviation (SD), and coefficient of variation
(CV) to evaluate the consistency and precision of the microarray prints

| | Diameter (μm) | Perimeter (μm) | Circularity¹ | Solidity² | Distance (μm)³ | Thickness (μm) |
|---|---|---|---|---|---|---|
| Mean | 166.0 | 549.7 | 0.901 | 0.975 | 411.8 | 5.21 |
| SD | 1.1 | 5.4 | 0.011 | 0.001 | 2.1 | 0.45 |
| CV | 0.66% | 0.98% | 1.17% | 0.11% | 0.51% | 8.65% |

${}^{1} Circularity = \frac{4 π \times Area}{{Perimeter}^{2}} ; a value of 1 indicates a perfect circle$ ${}^{2} Solidity = \frac{Area}{Convex Hull Area} ; values close to 1 indicate a very compact shape$ ³Horizontal and vertical distances between each pair of neighboring dots

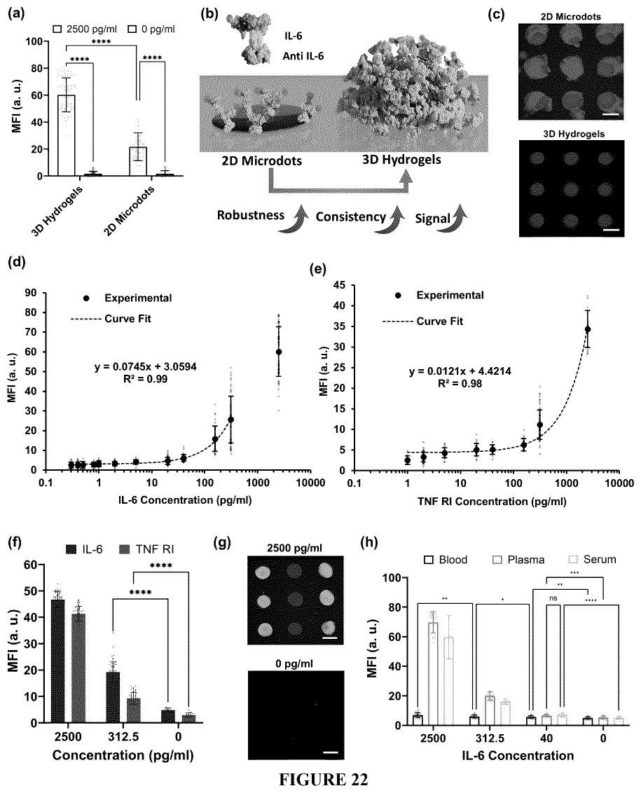

Multiplex Detection of Proinflammatory Biomarkers and IL-6 Detection in Complex Biological Fluids: To evaluate the biosensing advantages offered by 3D hydrogels, their signal intensity was compared to that of standard 2D microdot arrays. 2D microdot array fabrication involved a singular print of anti-IL-6 CAbs and EDC diluted in PBS, without the addition of BSA. While both techniques could detect IL-6, the 3D hydrogels outperformed the 2D microdots, to the order of three times more signal fluorescence (FIG. 22a). This enabled enhanced detection sensitivity (FIG. 22b). This is seen in fluorescence microscopy images, where a more definitive circular shape and brighter uniform fluorescence is observed with the 3D hydrogels. In comparison, the 2D microdots exhibited irregular morphologies and discrepancies in their fluorescence intensity (FIG. 22c).

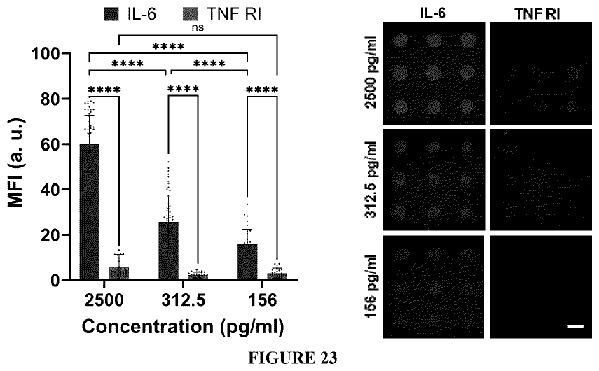

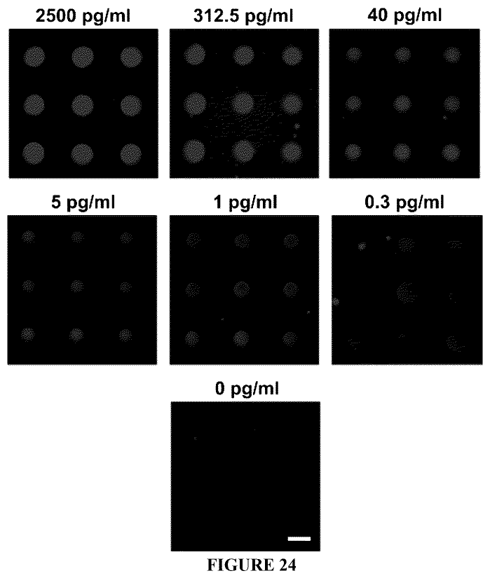

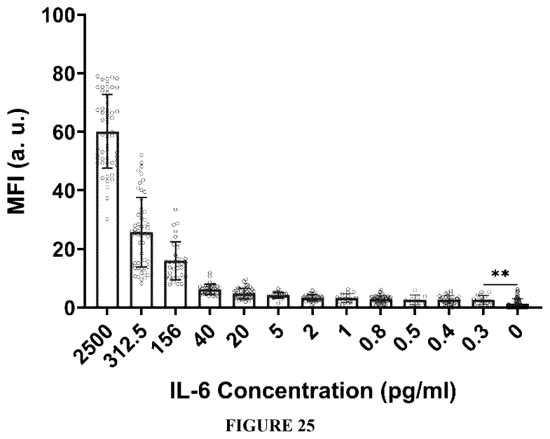

Next, the 3D hydrogels were employed within IFAs for the detection of IL-6 and TNF R1. The LOD of each proinflammatory biomarker as well as the linearity of detection were established through calibration curves, whereby the 3D microarrays were found to be highly sensitivity and specific (FIG. 22d-e, FIG. 23). For the detection of IL-6 in buffer, IL-6 concentrations between 0.10 pg/mL and 2500 pg/mL were tested, in which an LOD of 0.30 pg/mL and a linearity range of 0.30-312.5 pg/mL was obtained. Representative images for different concentrations of IL-6 are shown in FIG. 24, with a significant difference (P<0.01) evident between the 0.30 pg/mL and control samples (FIG. 25).

Compared to existing microfluidic, piezoelectric, and lateral flow detection systems reported in literature, this 3D hydrogel biosensing platform showed improved detection of IL-6, while being simpler to implement (Table 5) (22,23,36,69-75). Biofabrication protocols detailed in existing literature rely heavily on specialized substrates or microchip materials, as well as highly complex bioinks, composed of nanoparticles, electrochemical aptamers, semiconductive quantum dots, and magnetic beads (22,23,33,71,76,77). In comparison, this bio fabrication approach does not require these degrees of complexity, and can utilize easily accessible materials, such as glass substrates, and common, easy-to-use reagents, BSA and EDC. High repeatability, precision and ultra sensitivity was achieved, using an automated printer that is commercially viable, adding no additional effort or complexity with industry standards.

TABLE 5
IL-6 LODs reported in literature for various detection methods

| Title | LOD | | Strategy | Media | Ref. |
|---|---|---|---|---|---|
| Simultaneous immunoassay | 0.28 | pg/mL | piezoelectric | plasma | 23 |
| analysis of plasma IL-6 and | | | inkjet | | |
| TNF-α on a microchip | | | | | |
| Ultra-sensitive and semi- | 3.2 | pg/mL | vertical | serum | 69 |
| quantitative vertical flow assay | | | flow assay | | |
| for the rapid detection of | | | | | |
| Interleukin-6 in inflammatory | | | | | |
| diseases | | | | | |
| Rapid and sensitive detection of | 0.37 | pg/mL | lateral flow | serum | 22 |
| interleukin-6 in serum via time- | | | immunoassay | | |
| resolved lateral flow | | | | | |
| immunoassay | | | | | |
| Development of quantum dot- | 1.995 | pg/mL | quantum dot | serum | 70 |
| based fluorescence lateral flow | | | fluorescence | | |
| immunoassay strip for rapid and | | | lateral flow | | |
| quantitative detection of serum | | | immunoassay | | |
| interleukin-6 | | | | | |
| Fluorescence lateral flow | 0.9 | pg/mL | lateral flow | serum | 71 |
| immunoassay-based point-of- | | | immunoassay | | |
| care nano-diagnostics for | | | | | |
| orthopedic implant-associated | | | | | |
| infection | | | | | |
| Microfluidic magnetic analyte | 0.021 | pg/mL | microfluidic | serum | 72 |
| delivery technique for | | | device | | |
| separation, enrichment, and | | | | | |
| fluorescence detection of ultra- | | | | | |
| trace biomarkers | | | | | |
| Wave-shaped microfluidic chip | 16.25 | pg/mL | microfluidic | serum | 73 |
| assisted point-of-care testing for | | | device | | |
| accurate and rapid diagnosis of | | | | | |
| infections | | | | | |
| Inkjet-printed point-of-care | 6.3 | pg/mL | piezoelectric | serum | 36 |
| immunoassay on a nanoscale | 10.9 | pg/mL | inkjet | blood | |
| polymer brush enables sub- | | | | | |
| picomolar detection of analytes | | | | | |
| in blood | | | | | |
| Protein microarray for the | 6 | pg/mL | contact printed | serum | 74 |
| analysis of human melanoma | | | microarray | | |
| biomarkers | | | | | |
| Paper biosensors for detecting | 1.3 | pg/mL | nanoparticle paper- | blood | 75 |
| elevated IL-6 levels in blood | | | based device | | |
| and respiratory samples from | | | | | |
| COVID-19 patients | | | | | |

Furthermore, the inter-day and intra-day reproducibility of the IL-6 immunoassays were investigated to evaluate the precision of this biosensing platform. The MFI, SD, and CV values were calculated and are shown in Table 6. For a high IL-6 concentration of 2500 pg/mL, a CV value of 13% was obtained, which falls within the typically acceptable range of 10-15% for immunofluorescence assays (78,79). However, at a very low concentration of 0.8 pg/mL IL-6, the CV value was measured to be 37.5%. This higher CV at low concentrations is a common challenge in immunoassays, as signal variability tends to increase near the limit of detection. Despite this, the assay was able to detect significant differences between the control and low concentrations (p<0.01, as shown in FIG. 25), indicating that the assay can reliably distinguish these differences despite higher variability.

TABLE 6
Summary of mean fluorescent intensity (MFI), standard deviation
(SD), and coefficient of variation (CV) values for intra-day
(n = 18) and inter-day (n = 3) assays at three IL-6 concentrations

| IL-6 | Intra- | Intra- | Intra- | Inter- | Inter- | Inter- |
|---|---|---|---|---|---|---|
| Concentration | day | day | day | day | day | day |
| (pg/ml) | MFI | SD | CV | MFI | SD | CV |
| 2500 | 58.44 | 7.65 | 13.08% | 60.12 | 5.52 | 9.18% |
| 0.8 | 3.19 | 1.20 | 37.52% | 2.80 | 0.42 | 14.85% |

The inter-day repeatability was conducted over three days. The results indicated CV values of 9.18% and 14.85% for 2500 pg/mL and 0.8 pg/mL concentrations, respectively. The inter-day CV values of less than 15% for both high and low IL-6 concentrations confirm the robustness of the assay under varying day-to-day conditions.

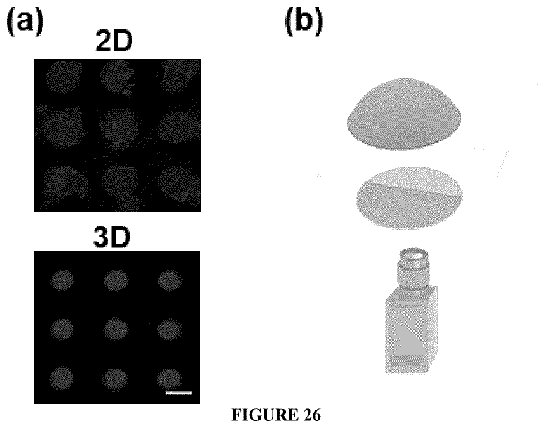

To assess the permeability of the hydrogels and the functionality of the antibodies embedded within, the fluorescence intensity per surface area was measured for 2D patterns and 3D domes after performing an IL-6 assay at a concentration of 2500 μg/mL (FIG. 26a). Notably, for the 3D samples, the fluorescence intensity detected by the microscope was a projection of the dome onto a 2D plane (FIG. 26b). The surface area of the 3D domes was calculated using their height and base diameter. The MFI2D was 20.0±10.6, while for the 3D samples (MFI3D), it was 58.3±12.7, nearly three times higher. This suggests that the antibodies within the bulk of the 3D domes are functional, contributing to the increased fluorescence.

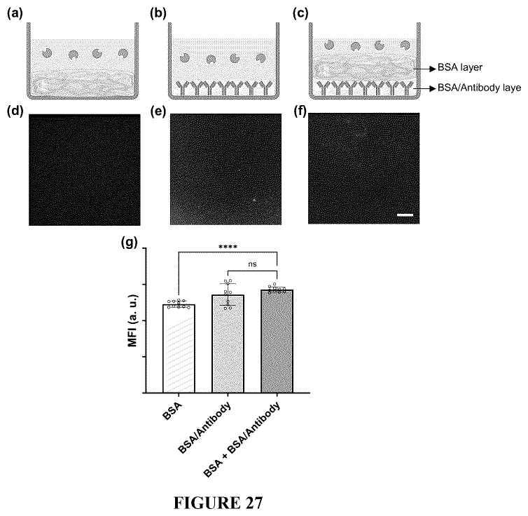

To further confirm the functionality of antibodies within the domes, an additional experiment was conducted using three bioinks (FIG. 27a-c): Bioink #1 (biotinylated antibody and 1% BSA), Bioink #2 (2 mg/mL EDC), and Bioink #3 (1% BSA). A glass-bottom well-plate was fluorosilanized using the modified protocol. For the negative control (BSA), a mixture of Bioink #2 and #3 was used to create a BSA gel layer. Positive controls (BSA/Antibody) were prepared by mixing Bioink #1 and #2. Test samples (BSA+BSA/Antibody) were created by sequentially adding Bioink #1 and #2, followed by an overlay of Bioink #2 and #3. After incubation with Streptavidin-Cy5 and subsequent washing, fluorescence imaging showed no significant difference in MFI between the positive controls and the BSA+BSA/Antibody samples, but both were significantly higher than the negative controls (FIG. 27d-g). These results confirm that the antibodies beneath the BSA layer remained functional and that the BSA gel's porosity provided sufficient permeability for the assay.

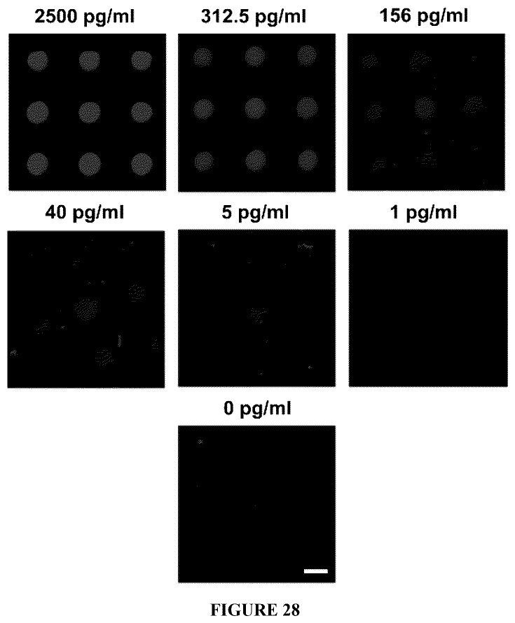

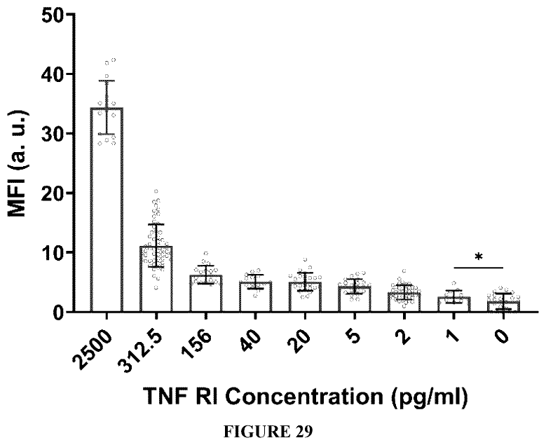

For the IFA of TNF R1, concentrations between 0.1 pg/mL-2500 μg/mL were tested, whereby an LOD of 1 pg/mL was determined, with a linearity range of 1-2500 pg/mL (FIG. 22c). FIG. 28 shows representative sample images for different TNF R1 concentrations, where a significant difference (P<0.05) between 1 pg/mL and the control can be observed (FIG. 29). To assess the specificity of the 3D hydrogels, it was tested if TNF R1 was detected at concentrations of 2500 pg/mL, 312.5 pg/mL and 0 pg/mL by anti-IL-6 CAbs-loaded 3D microarrays. Here, no statistically significant difference was observed between the TNF R1 concentrations, supporting the specificity of these hydrogels to IL-6 (FIG. 23).

Given the excellent sensitivity and specificity of the IL-6 and TNF antibody-loaded hydrogels, a multiplex microarray that consisted of both hydrogels was fabricated. This acted as a proof-of-concept for simultaneous proinflammatory biomarker detection (FIG. 22f-g). The multiplex microarray was constructed with a similar method as previous optimized hydrogel microarrays (composed of 2 L-1% BSA-2 mg/mL EDC hydrogels), where BSA bioink was printed first, followed by EDC bioink, for each layer. However, to allow for the detection of both IL-6 and TNF R1 in a single microarray, the preliminary printing stage involved printing of two different BSA bioinks, where one contained anti-IL-6 CAb while the other contained anti-TNF R1 CAb. Both bioinks were printed in alternating columns during the preliminary printing stage. The secondary EDC bioink was then printed along all the columns to induce hydrogel crosslinking. The multiplex microarray showed that IL-6 and TNF R1 were simultaneously detectable at 2500 pg/mL and 312.5 pg/mL, with significant difference (P<0.0001) compared to controls. The representative images in FIG. 22g depict the alternating column design of the multiplex microarray, where the first and third column showed TNF R1 detection, in green, while the second column showed IL-6 detection, in red.

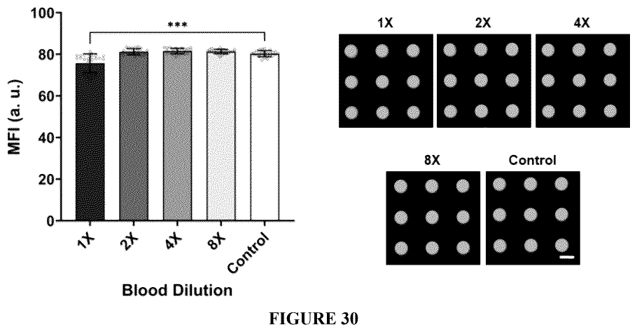

The platform was further utilized for the detection of IL-6 within citrated blood, plasma, and serum samples. Prior to performing IFA, the stability of the 3D microarrays in blood was evaluated. To this end, the microarrays containing BSA-FITC were incubated with blood diluted in HEPES for an hour on a shaker, by factors of 1×-8×. After washing, the samples were imaged and quantified (FIG. 30). Compared to the control that was incubated only in HEPES, there was a small reduction in the fluorescence intensity of microarrays incubated in 1× blood. Nevertheless, the intensity was still very high, demonstrating sufficient robustness.

To perform the IFA assay in biofluids, undiluted whole blood, plasma, and serum samples were spiked with IL-6 at concentrations of 2500 pg/mL, 312.5 pg/mL and 40 pg/mL. To perform the IFA assay in biofluids, undiluted whole blood, plasma, and serum samples were spiked with IL-6 at concentrations of 2500 pg/mL, 312.5 pg/mL and 40 pg/mL. These concentrations were chosen to show the clinical applicability of the platform in a wide range of IL-6 antigen concentrations. For instance, for high concentrations of IL-6 above the IL-6 clinically healthy range of 0-43.5 pg/mL, patients experience severe sepsis and widespread systemic infection (80). With the wide range of linearity, it can depict when individuals would be at the onset of infection, before the spread of infection, inflammation and cytokine storm have become systematic and too severe. These patients may or may not show febrile symptoms, making them difficult to detect and monitor through physical observation by a health professional, thus showing where the platform is most beneficial in clinical applications (81-87).

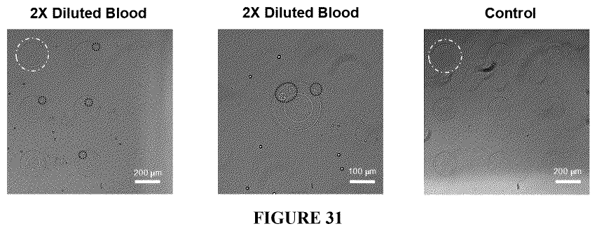

From the three samples, whole blood had the lowest MFI (FIG. 22h). This occurs because of the complexity of blood, where cellular components such as red blood cells, platelets, and immune cells are able to interfere with CAb target binding in the printed 3D hydrogels. This premise is supported by the demonstrated presence of blood cells at the microarray surface when incubated with 2× diluted blood (FIG. 31). Regardless, while the fluorescence signal was low, there was still a significant difference between 40 pg/ml IL-6 spiked in blood compared to the control samples (P<0.01). Compared to whole blood, the platform could accurately detect changes in IL-6 concentration in plasma and serum. A recovery of 93% was obtained for IL-6 spiked in plasma, demonstrating the applicability of the 3D hydrogels for clinical applications (Table 7).

TABLE 7
IL-6 concentrations utilized in the plasma
recovery test and resulting recovery %

| Original | Added | Found IL-6 | MFI CV | Recovery |
|---|---|---|---|---|
| (pg/ml) | (pg/ml) | (pg/ml) | (%)¹ | (%) |
| 0 | 40 | 37.17 | 12.78 | 93 |

¹Coefficient of variation of the mean fluorescence intensity

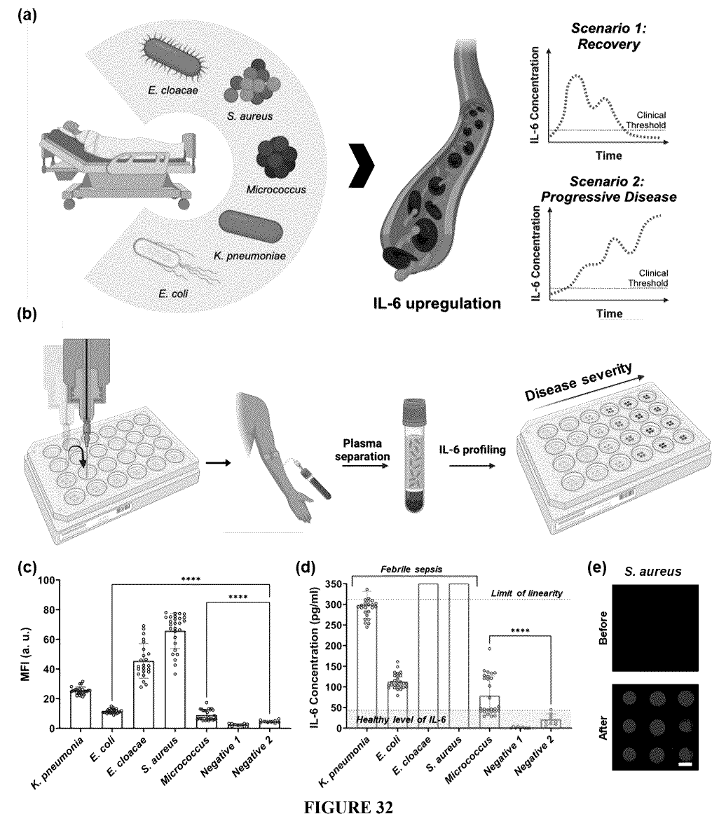

Detection of IL-6 in Clinical Septicemia Samples using 3D Proteinaceous Microarrays: In current clinical practices, the monitoring of septicemia (or sepsis) is done by observing the physical symptoms associated with sepsis and inflammation, known as febrile symptoms. This method is subjective, given that there is significant variation in the symptoms presented by septic patients. Literature points to the quantitative detection of proinflammatory biomarkers, specifically IL-6, as a method of measuring sepsis severity, since the overproduction of IL-6 cytokine yields cytokine storms, which severely damage tissues and organs within the body (22,84,88-90). By monitoring cytokine levels through IL-6 profiling, the 3D hydrogel microarrays can be integrated into a biosensing platform to measure the progression of bacteria-induced disease progression, and potential septicemia, as illustrated in FIG. 32a.

To assess efficacy for clinical detection, the ability of the 3D hydrogel microdots was evaluated to detect varying severities of inflammation through IL-6 biosensing, using clinical samples from patients suffering from bacteria-induced septicemia. FIG. 32b depicts the process of collecting whole blood samples from patients suffering with bacterial infection, followed by centrifugation to acquire plasma. For this study, blood samples collected from five patients were first cultured to determine the type of bacterial infection causing septicemia. The five bacterial infections were caused by Klebsiella pneumoniae (K. pneumonia), Escherichia coli (E. coli), Enterobacter cloacae (E. cloacae), Staphylococcus aureus (S. aureus), and Micrococcus, respectively. Four out of the five patients also experienced febrile symptoms associated with sepsis. Two bacteria negative samples from healthy donors were also collected as controls.

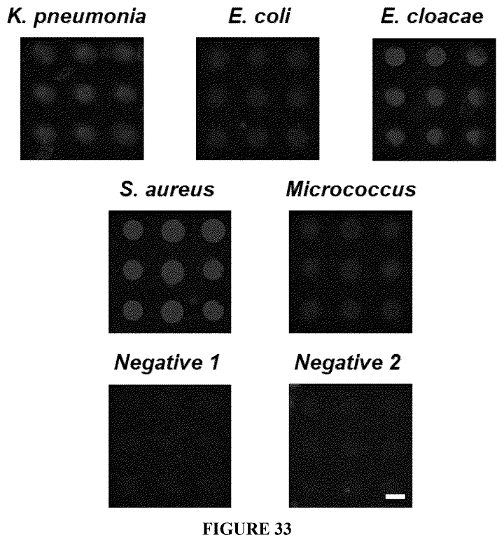

By running the sandwich-IFA with the patient and healthy donor blood plasma samples, it was determined that the 3D hydrogel microarray biosensing platform was effective in quantifying IL-6 concentration in clinical samples. The measured MFIs are shown in FIG. 32c, and the representative fluorescence images are illustrated in FIG. 33. According to the calibration curve (identified in FIG. 32d), the associated IL-6 concentration of each sample was calculated (FIG. 32d-c). The up-regulated IL-6 levels amongst the clinical plasma samples were greatly distinguishable and quantifiable compared to healthy controls. Patients with the highest IL-6 were distinguished, despite 4 out of the 5 patients showing similar febrile symptoms. The patients suffering from E. cloacae and S. aureus infection had IL-6 levels exceeding the limit of linearity from the calibration curve, at 312.5 pg/mL, indicative of a developing cytokine storm and tissue damage, requiring immediate therapeutic attention. Meanwhile, the patient with K. pneumonia had IL-6 levels approaching the limit of linearity, which was indicative of high severity and as such, would benefit from immediate adjustments to treatment. Contrarily, the E. coli and Micrococcus infected patients, with IL-6 levels between 50 pg/mL and 150 pg/mL, were not as high risk as the other three patients. Importantly, all diseased patient samples exhibited IL-6 levels above 43.5 pg/mL, which is the established threshold that distinguishes a normal and overexpressed state. Samples acquired from healthy controls, however, remained below 43.5 pg/mL, as expected (80). Detecting such subtle increases of IL-6 blood plasma levels in patient samples using an IFA platform provides a mechanism to delineate sepsis severity, enables real-time progression mapping of disease, and thus helps identify circumstances in which further clinical investigation is required. Collectively, such information stands to better inform clinical decision making (4,22,69,70,91). It was thus concluded that the 3D hydrogel biosensing platform effectively detects cytokine levels in clinical samples, offering value as a tool for the assessment of disease progression.

### Materials

The reagents and materials utilized for the experimental methods, such as surface functionalization, BSA-based hydrogel formation, and immunofluorescent assay preparation, include the following: trichloro(1H, 1H,2H,2H-perfluorooctyl) silane (TPFS) (Sigma-Aldrich, Oakville, ON, Canada), bovine serum albumin (BSA) (heat shock fraction, protease free, fatty acid free, essentially globulin free, pH 7, ≥98%, Sigma-Aldrich, Oakville, ON, Canada), 1-Ethyl-3-(3Dimethylaminopropyl) carbodiimide (EDC) (Sigma-Aldrich, Oakville, ON, Canada), 2-(N-Morpholino) ethanesulfonic acid (MES) (Sigma-Aldrich, Oakville, ON, Canada), phosphate-buffer silane (PBS) (R & D Systems, Minnesota, USA), N-Hydroxysuccinimide (NHS) (Solid, 98%, Sigma-Aldrich, Oakville, ON, Canada), L-Lysine hydrochloride (≥98%, natural, FG, Sigma-Aldrich, Oakville, ON, Canada), (3-Aminopropyl)triethoxysilane (APTES) (Sigma-Aldrich, Oakville, ON, Canada), IL-6 monoclonal antibody (MQ2-13A5, anti-IL-6 CAb) (ThermoFisher Scientific, ON, Canada), Human TNF R1 monoclonal antibody (TNFRSF1A, anti-TNF R1 CAb) (R & D Systems, Minnesota, USA), recombinant human (E. Coli derived) IL-6 (R & D Systems, Minnesota, USA), recombinant human sTNF R1 (TNFRSF1A Protein) (R & D Systems, Minnesota, USA), biotinylated IL-6 monoclonal antibody (MQ2-39C3, anti-IL-6 DAb) (ThermoFisher Scientific, ON, Canada), human TNF R1/TNFRSF1A biotinylated antibody (anti-TNF R1 DAb) (R & D Systems, Minnesota, USA), bovine serum albumin-fluorescein isothiocyanate conjugate (BSA-FITC) (Sigma-Aldrich, Oakville, ON, Canada), Streptavidin-Cy5 (Vector Laboratories, California, USA), Quantikine ELISA Wash Buffer (WB) (R & D Systems, Minnesota, USA), general assay diluent (ImmunoChemistry Technologies, California, USA), HEPES, Free Acid, Molecular Biology Grade—CAS 7365-45-9-Calbiochem (HEPES) (Sigma-Aldrich, Oakville, ON, Canada).

### Methods

Substrate Preparation and Functionalization: Before functionalization, a glass slide (75 mm×25 mm) was sonicated for 5 minutes with acetone and then carefully washed with 100% ethanol and dried with nitrogen gas to remove any traces of impurities. The surface was then CO2 plasma-treated for 3 minutes. Next, the substrate was immediately transferred to a vacuum desiccator to perform fluorosilanization through CVD using TPFS for 30 min at −0.08 MPa pressure. Afterwards, the surface with FS groups deposited was placed on a hot plate at 120.0° C. for 15 min to create FS SAMs. Subsequently, the substrate was sonicated in 100% ethanol for 10 minutes and dried with nitrogen gas. Contact angle measurements were taken before and after functionalizing using the Kruss Drop Shape Analyzer DSA30S.

Deconvolution of XPS and Quantification of Peak Area Percentage: XPS was conducted to determine functional group deposition at each substrate treatment step. An untreated glass substrate, a 3-minute CO2 plasma-treated substrate and a 30-minute TPFS CVD treated substrate were utilized. XPS data was gathered for each slide utilizing an Imaging and Scanning X-Ray Photoelectron Spectrometer. The raw data was then deconvoluted by performing Gaussian distribution of each functional group bond between the range of 280-296 eV (C1s range). The experimental or raw XPS data was compared to a curve of best fit, calculated through the summation of the peaks present. Atomic percent concentrations for each element as well as peak area percentages of each chemical bond were quantified after each stage.

Preparation and Optimization of Bioink: The first bioink was mixed with BSA, at concentrations of 1%, 1.5% or 2%, diluted in PBS. BSA-FITC, anti-IL-6 CAb or anti-TNF R1 CAb was added to the resulting bioink at 150 μg/mL. For the second bioink, EDC was prepared at concentration values of 1 mg/mL, 1.5 mg/mL, or 2 mg/mL, where solid EDC was diluted with MES at the pH of 4.5.

Printing and Immobilization of Proteinaceous Hydrogels: The sciFLEXARRAYER S3 SCIENION printer was used for the printing and immobilization of the 3D hydrogels onto the CO2 plasma-treated and FS SAM functionalized glass slide. A Piezo Dispense Capillaries nozzle (PDC 70, type 1) was utilized to generate droplets. The printer was set to a controlled humidity of 65% and a cooling temperature of 18° C. 10 droplets of bioink were printed in each dot, where the droplet volume and speed were 250-300 pL and 2.1-2.3 m/s respectively. The nozzle picked up and printed the BSA bioink first in a microarray pattern, for the primary printing step, followed by the secondary print of EDC solution directly on top, which encompassed one layer of the hydrogel. A 30-minute incubation followed. Additional layers were added by repeating the two-step printing approach described, followed incubated for another 30-minutes. To prevent the substrate from moving during printing, the printer is equipped with a porous stage connected to a vacuum pump. This setup keeps the substrate very stable on the stage throughout the printing process. After printing the first layer, the substrate on the stage was maintained for the incubation period. The printer chamber has humidity and temperature controllers, ensuring a precisely controlled atmosphere during incubation. This allows us to print the second layer without moving the substrate, thereby minimizing any potential positional shifts. Moreover, to address potential misalignment with droplet formation, the printer is equipped with a camera that can image the droplet with the same frequency as it is produced by the Piezo Dispense Capillary (PDC) nozzle. This enables us to monitor the droplet before it lands on the substrate. Prior to printing, the droplet formation was monitored and ensured it is completely aligned with the center of the nozzle. Slight adjustments can be made by changing the voltage and frequency using the printer's software. Once all layers were printed, the printed biosensing substrates were stored in a 100% humidity-controlled chamber, in a 4° C. fridge for a 24-hour incubation. For comparison of the 3D hydrogels, 2D microdots were created with a single printing step, using one bioink composed of anti-IL-6 CAb and EDC, diluted in PBS. The 2D microdots were then incubated for 30-minutes at 60% controlled humidity and 18° C., followed by 24-hour incubation at 4° C. in a 100% humidity-controlled chamber.

Stability Analysis After Washing: When preparing 3D hydrogels for stability analysis, the first bioink printed was composed of a combination of both BSA-FITC and BSA diluted in PBS. Concentrations of BSA tested for stability included 1%, 1.5% and 2%. This was followed by a secondary print with EDC diluted in MES, in which the concentrations tested included 1 mg/mL, 1.5 mg/mL and 2 mg/mL. These prints were conducted in either 1-layer, 2-layers, or 3-layers. Overall, 27 conditions were tested to ensure each possible combination of BSA concentration, EDC concentration and number of layers was accounted for. After a 24-hour incubation, the stability of each hydrogel condition was assessed in the following three stages: before wash, during a brief wash, and after an intense wash. The brief wash stage included two 20-30 second washes with DI water, while the intense wash included a 20-minute wash on a shaking incubator with wash buffer. Fluorescence microscopy imaging occurred with a Nikon ECLIPSE Ti2 Series Inverted Microscope, followed by signal quantification using ImageJ.

Non-Specific Attachment and Background Noise Analysis: When preparing 3D hydrogels for the non-specific attachment assessment, the proteinaceous hydrogels were printed in the same way, but with anti-IL-6 CAb, and with the same 27 conditions as in the stability analysis. The non-specific attachment assessment occurred after a 24-hour incubation. First, a superstructure, sonicated in ethanol for 5-minutes, was mounted onto the printed glass slide, and secured with clips. The superstructure was used to create wells for each 3D microarray of proteinaceous hydrogels, in which quick washes lasting 1-minute each were conducted with wash buffer (at volumes of 50 μL in each well). Each well then received 30 μL of Streptavidin-Cy5 diluted in general assay diluent with a concentration of 5 μL/1000 mL. Under controlled humidity of 100%, a 30-minute incubation in dark conditions followed on a shaking incubator at minimum shaking speed. The 3D hydrogels were imaged using fluorescence microscopy Nikon ECLIPSE Ti2 Series Inverted Microscope, and non-specific attachment was assessed by the fluorescence intensity (if observable) as well as signal quantification using ImageJ. Several approaches were tested involving lysine, APTES, BSA, and NHS treatment to reduce non-specific attachment in the hydrogel system. For lysine, following hydrogel formation and superstructure assembly, a 2% lysine solution was added to the wells, which were then incubated overnight in a humidified chamber inside the fridge to neutralize activated carboxyl groups. In APTES treatment, a 10% APTES solution diluted in PBS was printed on the spots after the initial BSA and EDC layers, aiming to react with the remaining carboxyl groups. In another approach, an extra BSA layer at a 2% concentration was printed without a subsequent EDC layer to further neutralize carboxyl groups. For the NHS protocol, after printing the BSA and EDC layers, a third layer of NHS was printed at a concentration of 120 mg/mL in MES buffer, with a 30-minute incubation between each layer.

Alignment Precision and Spot Morphology Characterization: ImageJ's particle analyzer function was used to calculate the diameter, perimeter, circularity, and solidity of the spots. Images were first converted to 8-bit images and the associated thresholds were adjusted to distinguish the printed spots from the background. The coordinates of the center point of each spot were determined the distances between the center points were calculated. Regarding the spot morphology in one-layer, two-layer, and three-layer, as moving the slide between layers for fluorescent imaging could introduce errors in the printing process, separate samples with different number of printed layers were used for imaging and quantification.

BSA-Based Hydrogel Characterization with SEM and Confocal Microscopy: After optimization, characterization was performed on the optimized BSA-based hydrogel conditions at 1-layer, 2-layer and 3-layers to determine surface topography and 3D microstructure features. SEM (TESCAN VEGA-II LSU) and confocal microscopy (Nikon A1R HD25) were performed. To prepare the samples before SEM, hydrogel samples were dehydrated using increasing concentrations of ethanol. For confocal microscopy, printed substrates were submerged in water and imaged at 40× magnification. Z-stack was performed, and thickness was quantified utilizing NIS-Elements software. Orthogonal projections were also constructed using NIS-Elements software.

IL-6, TNF R1 and Multiplex IFAs and Calibration Curve Plotting: From the list of 27 conditions analyzed for stability and nonspecific attachment, 2 L-1% BSA-2 mg/mL EDC conditions were determined to be optimized parameters. The optimized parameters were then used in the formation of proteinaceous hydrogels for the sandwich-IFA, in which CAbs were used in the BSA solution for the primary printing stages at values of 150 μg/mL. After hydrogels were printed, a 24-hour incubation occurred, and superstructure assembly was used to form wells onto the printed substrate. The IFAs conducted followed the standard sandwich ELISA structure. First, antigen diluted at various concentrations in general assay diluent was introduced and incubated for 1-hr. This was followed by application of a biotinylated detector antibody (DAb), diluted in general assay diluent with concentration of 5 μg/mL, for a similar 1-hr incubation. Finally, Streptavidin-Cy5 dye was diluted in general assay diluent, also at a concentration of 5 μg/mL, incubated for 30-minutes. Incubations for target analyte, DAb and Streptavidin-Cy5 all occurred at controlled 100% humidity and minimal shaking speed on a shaking incubator. Concentrations of each analyte tested, in pg/mL, are as follows: 2500, 312.5, 156, 40, 20, 5, 2, 1, 0.8, 0.5, 0.3, 0.1, 0. Furthermore, multiplex detection was also assessed, in which IL-6 and TNF R1 antigens were both mixed in solution at respective concentrations of 2500 pg/mL and 312.5 pg/mL, which were introduced to a combined anti-IL-6 and anti-TNF-RI CAb hydrogel multiplex microarray printed in alternating columns. For each IFA, fluorescence microscopy imaging was conducted using a Nikon ECLIPSE Ti2 Series Inverted Microscope. SNRs were also quantified using ImageJ, and linearity was plotted from these values in the form of a calibration curve. MFI was calculated and plotted. Statistical analysis was conducted using either T-Test or 2-way ANOVA (or mixed effects analysis with Geiser-Greenhouse corrections, p*<0.05, p**<0.01, p***<0.001, p****<0.0001).

Assessing the Antibody Functionality within the Hydrogel: A glass slide was first fluorosilanized using the same protocol employed for hydrogel printing. A multi-well structure with dimensions of 3.5×3.5 mm² was assembled to create a well-plate platform. Three different bioinks were prepared: Bioink #1 containing 90 μg/mL biotinylated detector antibody and 1% BSA (optimized concentration for printing), Bioink #2 consisting of 2 mg/mL EDC diluted in MES buffer at pH 4.5, and Bioink #3 containing 1% BSA in PBS. For the negative control, a mixture of Bioink #2 and #3 in a 1:1 ratio was used to create a BSA gel layer approximately 1.5 mm thick by adding 18 μL to each well. The positive control was prepared by mixing Bioink #1 and #2 in a 1:1 ratio and adding 3 μL of this solution to each well to form a BSA/Antibody layer. For the test samples, 3 μL of the Bioink #1 and #2 mixture was first added to each well and incubated for 30 minutes in a humidity chamber to form the initial BSA/Antibody layer. This was followed by adding 18 μL of the Bioink #2 and #3 mixture to create a second BSA gel layer without antibodies. All samples were incubated overnight at 4° C. in 100% humidity, consistent with the hydrogel printing protocol. Subsequently, the samples were incubated with Streptavidin-Cy5 at a concentration of 5 μg/mL for 30 minutes, followed by thorough washing. Fluorescence imaging was then performed, and the results were analyzed using ImageJ.

Preparation of Human Whole Blood, Plasma and Scrum: Human biological samples, provided by the Hamilton General Hospital at Hamilton Health Sciences, were utilized for IL-6 detection and determination of disease progression. For human biological samples, whole blood was gathered from healthy human donors. After a maximum of 1 hour, blood clots were removed from the whole blood and centrifuged at 500G for 10 minutes at minimal deceleration speed at room temperature. After centrifuging, the supernatant, in this scenario, plasma, was filtered and collected. A secondary centrifuging and supernatant collection step allowed for the separation of serum from the plasma. Human whole blood, plasma and serum were all assessed to determine the ideal medium for the sandwich-IFA. As an additional test, human whole blood was diluted in HEPES at values of 1×, 2×, 4× and 8× dilution. Brightfield images of 2× diluted whole blood were taken to observe red blood cell attachment and interference. Afterwards, each printed microarray on the substrate was submerged with non-diluted human whole blood, plasma or serum, as well as all human whole blood dilutions, and then incubated within a humidity-controlled chamber at approximately 100% relative humidity. The non diluted whole blood, plasma, and serum were then spiked with IL-6 antigen at concentrations of 2500 pg/mL, 312.5 pg/mL, 40 pg/mL and 0 pg/mL, and substrates with the printed hydrogel microarrays were again submerged, as described. The detection steps of the sandwich IFAs were conducted for all hydrogel microarrays using human biological samples, followed by fluorescence microscopy imaging and MFI calculations. From these results, plasma was deemed most suitable plasma, and a recovery test was performed, whereby plasma spiked with 40 pg/mL IL-6 antigen was compared to non-spiked plasma, with assumed IL-6 concentration of 20 pg/mL as well as buffer spiked with 60 pg/mL IL-6 antigen and buffer only. Equation 1 describes the recovery test calculations performed. Statistical analysis was conducted using 2-way ANOVA (or mixed effects analysis with Geiser-Greenhouse corrections, p*<0.05, p**<0.01, p***<0.001, p****<0.0001).

$$Recovery \% = \frac{{c}_{spiked} - {c}_{original ( unspiked )}}{{c}_{a d d e d}} ( 100 \% ) \qquad ( 1 )$$

Human Bacterial-Induced Sepsis Monitoring Using IL-6 Induced Fluorescence: Whole blood was drawn from 7 human donors provided by the Hamilton General Hospital at Hamilton Health Sciences, 5 of which showed febrile signs and were found to have bacterial infections (K. Pneumonia, E. coli, E. cloacae, S. aureus, and micrococcus) by blood culturing, citrating, and clot removal. The remaining two donors did not have febrile symptoms and no bacterial infection. The whole blood was then centrifuged, and plasma was collected, similar to previously described. Microarrays were printed in treated 96 glass-bottom well-plates rather than slides, and a sandwich-IFA was performed on each of the 7 platelet poor plasma samples. To conduct this sandwich-IFA, and to detect the target IL-6, the hydrogel microarrays printed on each well of the glass well-plates were submerged in the patient samples for one-hour and incubated at approximately 100% relative humidity. Fluorescence images were taken using a Nikon ECLIPSE Ti2 Series Inverted Microscope after sandwich-IFA completion, SNR was calculated with ImageJ, and MFI was quantified. Using the predetermined calibration curve and equation of linearity for IL-6 (linearity equation: y=0.0745×+3.0594), IL-6 concentrations were determined for each plasma sample.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### CITATIONS

(1) Qin, J.; Wang, W.; Gao, L.; Yao, S. Q. Emerging Biosensing and Transducing Techniques for Potential Applications in Point-of-Care Diagnostics. Chem Sci 2022, 13 (10), 2857-2876. https://doi.org/10.1039/DISC06269G.
(2) Ma, L.; Abugalyon, Y.; Li, X. Multicolorimetric ELISA Biosensors on a Paper/Polymer Hybrid Analytical Device for Visual Point-of-Care Detection of Infection Diseases. Anal Bioanal Chem 2021, 413 (18), 4655-4663. https://doi.org/10.1007/s00216-021-03359-8.
(3) Shakeri, A.; Imani, S. M.; Chen, E.; Yousefi, H.; Shabbir, R.; Didar, T. F. Plasma-Induced Covalent Immobilization and Patterning of Bioactive Species in Microfluidic Devices. Lab Chip 2019, 19 (18), 3104-3115. https://doi.org/10.1039/C9 LC00364A.
(4) Shakeri, A.; Jarad, N. A.; Terryberry, J.; Khan, S.; Leung, A.; Chen, S.; Didar, T. F. Antibody Micropatterned Lubricant-Infused Biosensors Enable Sub-Picogram Immunofluorescence Detection of Interleukin 6 in Human Whole Plasma. Small 2020, 16 (45), 2003844. https://doi.org/10.1002/smll.202003844.
(5) Iswardy, E.; Tsai, T. C.; Cheng, I. F.; Ho, T. C.; Perng, G. C.; Chang, H. C. A Bead-Based Immunofluorescence-Assay on a Microfluidic Dielectrophoresis Platform for Rapid Dengue Virus Detection. Biosens Bioelectron 2017, 95, 174-180. https://doi.org/10.1016/j.bios.2017.04.011.
(6) Kakkar, S.; Gupta, P.; Kumar, N.; Kant, K. Progress in Fluorescence Biosensing and Food Safety towards Point-of-Detection (POD) System. Biosensors (Basel) 2023, 13 (2), 249. https://doi.org/10.3390/bios 13020249.
(7) Kusnezow, W.; Jacob, A.; Walijew, A.; Diehl, F.; Hoheisel, J. D. Antibody Microarrays: An Evaluation of Production Parameters. Proteomics 2003, 3 (3), 254-264. https://doi.org/10.1002/pmic.200390038.
(8) Lichade, K. M.; Jiang, Y.; Pan, Y. Hierarchical Nano/Micro-Structured Surfaces With High Surface Arca/Volume Ratios. J Manuf Sci Eng 2021, 143 (8). https://doi.org/10.1115/1.4049850.
(9) Khan, S.; Burciu, B.; Filipe, C. D. M.; Li, Y.; Dellinger, K.; Didar, T. F. DNAzyme-Based Biosensors: Immobilization Strategies, Applications, and Future Prospective. ACS Nano 2021, 15 (9), 13943-13969. https://doi.org/10.1021/acsnano.1c04327.
(10) Didar, T. F.; Li, K.; Veres, T.; Tabrizian, M. Separation of Rare Oligodendrocyte Progenitor Cells from Brain Using a High-Throughput Multilayer Thermoplastic-Based Microfluidic Device. Biomaterials 2013, 34 (22), 5588-5593. https://doi.org/10.1016/j.biomaterials.2013.04.014.
(11) Prasad, A.; Khan, S.; Monteiro, J. K.; Li, J.; Arshad, F.; Ladouceur, L.; Tian, L.; Shakeri, A.; Filipe, C. D. M.; Li, Y.; Didar, T. F. Advancing In Situ Food Monitoring through a Smart Lab-in-a-Package System Demonstrated by the Detection of Salmonella in Whole Chicken. Advanced Materials 2023, 35 (40). https://doi.org/10.1002/adma.202302641.
(12) Yousefi, H.; Samani, S. E.; Khan, S.; Prasad, A.; Shakeri, A.; Li, Y.; Filipe, C. D. M.; Didar,
T. F. LISzyme Biosensors: DNAzymes Embedded in an Anti-Biofouling Platform for Hands-Free Real-Time Detection of Bacterial Contamination in Milk. ACS Nano 2022, 16 (1), 29-37. https://doi.org/10.1021/acsnano.1c05766.
(13) Didar, T. F.; Tabrizian, M. Generating Multiplex Gradients of Biomolecules for Controlling Cellular Adhesion in Parallel Microfluidic Channels. Lab Chip 2012, 12 (21), 4363. https://doi.org/10.1039/c2lc40233e.
(14) Yousefi, H.; Su, H.; Ali, M.; Filipe, C. D. M.; Didar, T. F. Producing Covalent Microarrays of Amine-Conjugated DNA Probes on Various Functional Surfaces to Create Stable and Reliable Biosensors. Adv Mater Interfaces 2018, 5 (16). https://doi.org/10.1002/admi.201800659.
(15) Fayad, L.; Keating, M. J.; Reuben, J. M.; O'Brien, S.; Lee, B. N.; Lerner, S.; Kurzrock, R. Interleukin-6 and Interleukin-10 Levels in Chronic Lymphocytic Leukemia: Correlation with Phenotypic Characteristics and Outcome. Blood 2001, 97 (1), 256-263. https://doi.org/10.1182/blood.V97.1.256.
(16) Knüpfer, H.; Preiß, R. Significance of Interleukin-6 (IL-6) in Breast Cancer (Review). Breast Cancer Res Treat 2007, 102 (2), 129-135. https://doi.org/10.1007/s10549-006-9328-3.
(17) George, D. J.; Halabi, S.; Shepard, T. F.; Sanford, B.; Vogelzang, N. J.; Small, E. J.; Kantoff, P. W. The Prognostic Significance of Plasma Interleukin-6 Levels in Patients with Metastatic Hormone-Refractory Prostate Cancer: Results from Cancer and Leukemia Group B 9480. Clinical Cancer Research 2005, 11 (5), 1815-1820. https://doi.org/10.1158/1078-0432.CCR-04-1560.
(18) Plante, M.; Rubin, S. C.; Wong, G. Y.; Federici, M. G.; Finstad, C. L.; Gastl, G. A. Interleukin-6 Level in Serum and Ascites as a Prognostic Factor in Patients with Epithelial Ovarian Cancer. Cancer 1994, 73 (7), 1882-1888. https://doi.org/10.1002/1097-0142(19940401)73:7<1882::aid-cncr2820730718>3.0.co; 2-r.
(19) Chung, Y.; Chang, Y. Serum Interleukin-6 Levels Reflect the Disease Status of Colorectal Cancer. J Surg Oncol 2003, 83 (4), 222-226. https://doi.org/10.1002/jso. 10269.
(20) Chen, X.; Zhao, B.; Qu, Y.; Chen, Y.; Xiong, J.; Feng, Y.; Men, D.; Huang, Q.; Liu, Y.; Yang, B.; Ding, J.; Li, F. Detectable Serum Severe Acute Respiratory Syndrome Coronavirus 2 Viral Load (RNAemia) Is Closely Correlated With Drastically Elevated Interleukin 6 Level in Critically Ill Patients With Coronavirus Disease 2019. Clinical Infectious Diseases 2020, 71 (8), 1937-1942. https://doi.org/10.1093/cid/ciaa449.
(21) Röckendorf, N.; Ramaker, K.; Gaede, K.; Tappertzhofen, K.; Lunding, L.; Wegmann, M.; Horbert, P.; Weber, K.; Frey, A. Parallel Detection of Multiple Biomarkers in a Point-of-Care-Competent Device for the Prediction of Exacerbations in Chronic Inflammatory Lung Disease. Sci Rep 2024, 14 (1), 12830. https://doi.org/10.1038/s41598-024-62784-8.
(22) Huang, D.; Ying, H.; Jiang, D.; Liu, F.; Tian, Y.; Du, C.; Zhang, L.; Pu, X. Rapid and Sensitive Detection of Interleukin-6 in Serum via Time-Resolved Lateral Flow Immunoassay. Anal Biochem 2020, 588, 113468. https://doi.org/10.1016/j.ab.2019.113468.
(23) Abe, K.; Hashimoto, Y.; Yatsushiro, S.; Yamamura, S.; Bando, M.; Hiroshima, Y.; Kido, J.; Tanaka, M.; Shinohara, Y.; Ooie, T.; Baba, Y.; Kataoka, M. Simultaneous Immunoassay Analysis of Plasma IL-6 and TNF-α on a Microchip. PLOS One 2013, 8 (1), c53620. https://doi.org/10.1371/journal.pone.0053620.
(24) Didar, T. F.; Li, K.; Tabrizian, M.; Veres, T. High Throughput Multilayer Microfluidic Particle Separation Platform Using Embedded Thermoplastic-Based Micropumping. Lab Chip 2013, 13 (13), 2615. https://doi.org/10.1039/c3lc50181g.
(25) Bot, V. A.; Shakeri, A.; Weitz, J. I.; Didar, T. F. A Vascular Graft On-a-Chip Platform for Assessing the Thrombogenicity of Vascular Prosthesis and Coatings with Tunable Flow Surface and Conditions. Adv Funct Mater 2022, 32 (41). https://doi.org/10.1002/adfm.202205078.
(26) Stack, E. C.; Wang, C.; Roman, K. A.; Hoyt, C. C. Multiplexed Immunohistochemistry, Imaging, and Quantitation: A Review, with an Assessment of Tyramide Signal Amplification, Multispectral Imaging and Multiplex Analysis. Methods 2014, 70 (1), 46-58. https://doi.org/10.1016/j.ymeth.2014.08.016.
(27) Charles, P. T.; Goldman, E. R.; Rangasammy, J. G.; Schauer, C. L.; Chen, M. S.; Taitt, C. R. Fabrication and Characterization of 3D Hydrogel Microarrays to Measure Antigenicity and Antibody Functionality for Biosensor Applications. Biosens Bioelectron 2004, 20 (4), 753-764. https://doi.org/10.1016/j.bios.2004.04.007.
(28) Tian, L.; He, L.; Jackson, K.; Saif, A.; Khan, S.; Wan, Z.; Didar, T. F.; Hosseinidoust, Z. Self-Assembling Nanofibrous Bacteriophage Microgels as Sprayable Antimicrobials Targeting Multidrug-Resistant Bacteria. Nat Commun 2022, 13 (1), 7158. https://doi.org/10.1038/s41467-022-34803-7.
(29) ILKHANIZADEH, S.; TEIXEIRA, A.; HERMANSON, O. Inkjet Printing of Macromolecules on Hydrogels to Steer Neural Stem Cell Differentiation. Biomaterials 2007, 28 (27), 3936-3943. https://doi.org/10.1016/j.biomaterials.2007.05.018.
(30) Li, L.; Pan, L.; Ma, Z.; Yan, K.; Cheng, W.; Shi, Y.; Yu, G. All Inkjet-Printed Amperometric Multiplexed Biosensors Based on Nanostructured Conductive Hydrogel Electrodes. Nano Lett 2018, 18 (6), 3322-3327. https://doi.org/10.1021/acs.nanolett.8b00003.
(31) Al Sulaiman, D.; Shapiro, S. J.; Gomez-Marquez, J.; Doyle, P. S. High-Resolution Patterning of Hydrogel Sensing Motifs within Fibrous Substrates for Sensitive and Multiplexed Detection of Biomarkers. ACS Sens 2021, 6 (1), 203-211. https://doi.org/10.1021/acssensors.0c02121.
(32) Yodmongkol, S.; Sutapun, B.; Praphanphoj, V.; Srikhirin, T.; Brandstetter, T.; Rühe, J. Fabrication of Protein Microarrays for Alpha Fetoprotein Detection by Using a Rapid Photo-Immobilization Process. Res Sens Biosensing 2016, 7, 95-99. https://doi.org/10.1016/j.sbsr.2016.01.010.
(33) Stumpf, A.; Brandstetter, T.; Hübner, J.; Rühe, J. Hydrogel Based Protein Biochip for Parallel Detection of Biomarkers for Diagnosis of a Systemic Inflammatory Response Syndrome (SIRS) in Human Scrum. PLOS One 2019, 14 (12), c0225525. https://doi.org/10.1371/journal.pone.0225525.
(34) Summers, A. J.; Devadhasan, J. P.; Gu, J.; Montgomery, D. C.; Fischer, B.; Gates-Hollingsworth, M. A.; Pflughoeft, K. J.; Vo-Dinh, T.; AuCoin, D. P.; Zenhausern, F. Optimization of an Antibody Microarray Printing Process Using a Designed Experiment. ACS Omega 2022, 7 (36), 32262-32271. https://doi.org/10.1021/acsomega.2c03595.
(35) Zub, K.; Hoeppener, S.; Schubert, U. S. Inkjet Printing and 3D Printing Strategies for Biosensing, Analytical, and Diagnostic Applications. Advanced Materials 2022, 34 (31), 2105015. https://doi.org/10.1002/adma.202105015.
(36) Joh, D. Y.; Hucknall, A. M.; Wei, Q.; Mason, K. A.; Lund, M. L.; Fontes, C. M.; Hill, R. T.; Blair, R.; Zimmers, Z.; Achar, R. K.; Tseng, D.; Gordan, R.; Freemark, M.; Ozcan, A.; Chilkoti, A. Inkjet-Printed Point-of-Care Immunoassay on a Nanoscale Polymer Brush Enables Subpicomolar Detection of Analytes in Blood. Proceedings of the National Academy of Sciences 2017, 114 (34). https://doi.org/10.1073/pnas. 1703200114.
(37) Sundriyal, P.; Bhattacharya, S. Inkjet-Printed Sensors on Flexible Substrates; 2018; pp 89-113. https://doi.org/10.1007/978-981-10-7751-7_5.
(38) Wang, X.; Zhang, M.; Zhang, L.; Xu, J.; Xiao, X.; Zhang, X. Inkjet-Printed Flexible Sensors: From Function Materials, Manufacture Process, and Applications Perspective. Mater Today Commun 2022, 31, 103263. https://doi.org/10.1016/j.mtcomm.2022.103263.
(39) Du, Z.; Zhou, H.; Yu, X.; Han, Y. Controlling the Polarity and Viscosity of Small Molecule Ink to Suppress the Contact Line Receding and Coffee Ring Effect during Inkjet Printing. Colloids Surf A Physicochem Eng Asp 2020, 602, 125111. https://doi.org/10.1016/j.colsurfa.2020.125111.
(40) Uddin, M. J.; Hassan, J.; Douroumis, D. Thermal Inkjet Printing: Prospects and Applications in the Development of Medicine. Technologies (Basel) 2022, 10 (5), 108. https://doi.org/10.3390/technologies10050108.
(41) Li, J.; Rossignol, F.; Macdonald, J. Inkjet Printing for Biosensor Fabrication: Combining Chemistry and Technology for Advanced Manufacturing. Lab Chip 2015, 15 (12), 2538-2558. https://doi.org/10.1039/C5 LC00235D.
(42) Lazar-Poloczek, E.; Romuk, E.; Jacheć, W.; Stanek, W.; Stanek, B.; Szołtysik, M.; Techmański, T.; Hasterok, M.; Wojciechowska, C. Levels of TNF-α and Soluble TNF Receptors in Normal-Weight, Overweight and Obese Patients with Dilated Non-Ischemic Cardiomyopathy: Does Anti-TNF Therapy Still Have Potential to Be Used in Heart Failure Depending on BMI? Biomedicines 2022, 10 (11), 2959. https://doi.org/10.3390/biomedicines10112959.
(43) Geering, B.; Gurzeler, U.; Federzoni, E.; Kaufmann, T.; Simon, H. U. A Novel TNFR1-Triggered Apoptosis Pathway Mediated by Class IA PI3Ks in Neutrophils. Blood 2011, 117 (22), 5953-5962. https://doi.org/10.1182/blood-2010-11-322206.
(44) Salai, K. H. T.; Wu, L. Y.; Chong, J. R.; Chai, Y. L.; Gyanwali, B.; Robert, C.; Hilal, S.; Venkatasubramanian, N.; Dawe, G. S.; Chen, C. P.; Lai, M. K. P. Elevated Soluble TNF-Receptor 1 in the Serum of Predementia Subjects with Cerebral Small Vessel Disease. Biomolecules 2023, 13 (3), 525. https://doi.org/10.3390/biom13030525.
(45) Boehme, A. K.; McClure, L. A.; Zhang, Y.; Luna, J. M.; Del Brutto, O. H.; Benavente, O. R.; Elkind, M. S. V. Inflammatory Markers and Outcomes After Lacunar Stroke. Stroke 2016, 47 (3), 659-667. https://doi.org/10.1161/STROKEAHA.115.012166.
(46) Dhamoon, M. S.; Cheung, Y. K.; Moon, Y. P.; Wright, C. B.; Willey, J. Z.; Sacco, R. L.; Elkind, M. S. V. Association Between Serum Tumor Necrosis Factor Receptor 1 and Trajectories of Functional Status. Am J Epidemiol 2017, 186 (1), 11-20. https://doi.org/10.1093/aje/kwx035.
(47) Didar, T.; Shakeri, A.; Yousefi, H. Lubricant-Infused Surface Biosensing Interface, Methods of Making and Uses Thereof. US20230349893A1, 2021.
(48) Shakeri, A.; Yousefi, H.; Jarad, N. A.; Kullab, S.; Al-Mfarej, D.; Rottman, M.; Didar, T. F. Contamination and Carryover Free Handling of Complex Fluids Using Lubricant-Infused Pipette Tips. Sci Rep 2022, 12 (1), 14486. https://doi.org/10.1038/s41598-022-18756-x.
(49) Shakeri, A.; Jarad, N. A.; Leung, A.; Soleymani, L.; Didar, T. F. Biofunctionalization of Glass- and Paper-Based Microfluidic Devices: A Review. Adv Mater Interfaces 2019, 6 (19). https://doi.org/10.1002/admi.201900940.
(50) Law, K. Y. Definitions for Hydrophilicity, Hydrophobicity, and Superhydrophobicity: Getting the Basics Right. J Phys Chem Lett 2014, 5 (4), 686-688. https://doi.org/10.1021/jz402762h.
(51) Abu Jarad, N.; Rachwalski, K.; Bayat, F.; Khan, S.; Shakeri, A.; MacLachlan, R.; Villegas, M.; Brown, E. D.; Soleymani, L.; Didar, T. F. An Omniphobic Spray Coating Created from Hierarchical Structures Prevents the Contamination of High-Touch Surfaces with Pathogens. Small 2023, 19 (12). https://doi.org/10.1002/smll.202205761.
(52) Kasapgil, E.; Badv, M.; Cantú, C. A.; Rahmani, S.; Erbil, H. Y.; Anac Sakir, I.; Weitz, J. I.; Hosseini-Doust, Z.; Didar, T. F. Polysiloxane Nanofilaments Infused with Silicone Oil Prevent Bacterial Adhesion and Suppress Thrombosis on Intranasal Splints. ACS Biomater Sci Eng 2021, 7 (2), 541-552. https://doi.org/10.1021/acsbiomaterials.0c01487.
(53) Yan, C.; Wu, F.; Jernigan, R. L.; Dobbs, D.; Honavar, V. Characterization of Protein-Protein Interfaces. Protein J 2008, 27 (1), 59-70. https://doi.org/10.1007/s10930-007-9108-X.
(54) Gao, X.; Zhang, Y.; Liu, Y. Temperature-Dependent Hygroscopic Behaviors of Atmospherically Relevant Water-Soluble Carboxylic Acid Salts Studied by ATR-FTIR Spectroscopy. Atmos Environ 2018, 191, 312-319. https://doi.org/10.1016/j.atmosenv.2018.08.021.
(55) Ge, Y.; Wang, H.; Xue, J.; Jiang, J.; Liu, Z.; Liu, Z.; Li, G.; Zhao, Y. Programmable Humidity-Responsive Actuation of Polymer Films Enabled by Combining Shape Memory Property and Surface-Tunable Hygroscopicity. ACS Appl Mater Interfaces 2021, 13 (32), 38773-38782. https://doi.org/10.1021/acsami.lc11862.
(56) Ahmad, R.; Wolfbeis, O. S.; Hahn, Y. B.; Alshareef, H. N.; Torsi, L.; Salama, K. N. Deposition of Nanomaterials: A Crucial Step in Biosensor Fabrication. Mater Today Commun 2018, 17, 289-321. https://doi.org/10.1016/j.mtcomm.2018.09.024.
(57) Li, X.; Liu, B.; Pei, B.; Chen, J.; Zhou, D.; Peng, J.; Zhang, X.; Jia, W.; Xu, T. Inkjet Bioprinting of Biomaterials. Chem Rev 2020, 120 (19), 10793-10833. https://doi.org/10.1021/acs.chemrev.0c00008.
(58) Saunders, R. E.; Derby, B. Inkjet Printing Biomaterials for Tissue Engineering: Bioprinting. International Materials Reviews 2014, 59 (8), 430-448. https://doi.org/10.1179/1743280414Y.0000000040.
(59) Cornilescu, G.; Hu, J. S.; Bax, A. Identification of the Hydrogen Bonding Network in a Protein by Scalar Couplings. J Am Chem Soc 1999, 121 (12), 2949-2950. https://doi.org/10.1021/ja9902221.
(60) Petters, S. S.; Pagonis, D.; Claflin, M. S.; Levin, E. J. T.; Petters, M. D.; Ziemann, P. J.; Kreidenweis, S. M. Hygroscopicity of Organic Compounds as a Function of Carbon Chain Length and Carboxyl, Hydroperoxy, and Carbonyl Functional Groups. J Phys Chem A 2017, 121 (27), 5164-5174. https://doi.org/10.1021/acs.jpca.7b04114.
(61) Yang, M.; Chen, D.; Hu, J.; Zheng, X.; Lin, Z. J.; Zhu, H. The Application of Coffee-Ring Effect in Analytical Chemistry. TrAC Trends in Analytical Chemistry 2022, 157, 116752. https://doi.org/10.1016/j.trac.2022.116752.
(62) He, P.; Derby, B. Controlling Coffee Ring Formation during Drying of Inkjet Printed 2D Inks. Adv Mater Interfaces 2017, 4 (22). https://doi.org/10.1002/admi.201700944.
(63) Lichtenberg, J. Y.; Ling, Y.; Kim, S. Non-Specific Adsorption Reduction Methods in Biosensing. Sensors 2019, 19 (11), 2488. https://doi.org/10.3390/s19112488.
(64) Mädler, S.; Bich, C.; Touboul, D.; Zenobi, R. Chemical Cross-linking with NHS Esters: A Systematic Study on Amino Acid Reactivities. Journal of Mass Spectrometry 2009, 44 (5), 694-706. https://doi.org/10.1002/jms.1544.
(65) Kimura, S.; Komiyama, T.; Masuzawa, T.; Yokoya, M.; Oyoshi, T.; Yamanaka, M. Bovine Serum Albumin Hydrogel Formation: P H Dependence and Rheological Analyses. Chem Pharm Bull (Tokyo) 2023, 71 (3), c22-00758. https://doi.org/10.1248/cpb.c22-00758.
(66) Khanna, S.; Singh, A. K.; Behera, S. P.; Gupta, S. Thermoresponsive BSA Hydrogels with Phase Tunability. Materials Science and Engineering: C 2021, 119, 111590. https://doi.org/10.1016/j.msec.2020.111590.
(67) Xia, T.; Jiang, X.; Deng, L.; Yang, M.; Chen, X. Albumin-Based Dynamic Double CrossLinked Hydrogel with Self-Healing Property for Antimicrobial Application. Colloids Surf B Biointerfaces 2021, 208, 112042. https://doi.org/10.1016/j.colsurfb.2021.112042.
(68) Liu, D.; Zhao, S.; Jiang, Y.; Gao, C.; Wu, Y.; Liu, Y. Biocompatible Dual Network Bovine Serum Albumin-Loaded Hydrogel-Accelerates Wound Healing. Eur Polym J 2023, 185, 111820. https://doi.org/10.1016/j.eurpolymj.2023.111820.
(69) Lei, R.; Arain, H.; Obaid, M.; Sabhnani, N.; Mohan, C. Ultra-Sensitive and Semi-Quantitative Vertical Flow Assay for the Rapid Detection of Interleukin-6 in Inflammatory Diseases. Biosensors (Basel) 2022, 12 (9), 756. https://doi.org/10.3390/bios12090756.
(70) Tang, J.; Wu, L.; Lin, J.; Zhang, E.; Luo, Y. Development of Quantum Dot-based Fluorescence Lateral Flow Immunoassay Strip for Rapid and Quantitative Detection of Serum Interleukin-6. J Clin Lab Anal 2021, 35 (5). https://doi.org/10.1002/jcla.23752.
(71) Borse, V.; Srivastava, R. Fluorescence Lateral Flow Immunoassay Based Point-of-Care Nanodiagnostics for Orthopedic Implant-Associated Infection. Sens Actuators B Chem 2019, 280, 24-33. https://doi.org/10.1016/j.snb.2018.10.034.
(72) Yang, J.; Xiao, X.; Xia, L.; Li, G.; Shui, L. Microfluidic Magnetic Analyte Delivery Technique for Separation, Enrichment, and Fluorescence Detection of Ultratrace Biomarkers. Anal Chem 2021, 93 (23), 8273-8280. https://doi.org/10.1021/acs.analchem.1c01130.
(73) Yin, B. F.; Wan, X. H.; Yang, M. Z.; Qian, C. C.; Sohan, A. S. M. M. F. Wave-Shaped Microfluidic Chip Assisted Point-of-Care Testing for Accurate and Rapid Diagnosis of Infections. Mil Med Res 2022, 9 (1), 8. https://doi.org/10.1186/s40779-022-00368-1.
(74) Domnanich, P.; Sauer, U.; Pultar, J.; Preininger, C. Protein Microarray for the Analysis of Human Melanoma Biomarkers. Sens Actuators B Chem 2009, 139 (1), 2-8. https://doi.org/10.1016/j.snb.2008.06.043.
(75) Adrover-Jaume, C.; Alba-Patiño, A.; Clemente, A.; Santopolo, G.; Vaquer, A.; Russell, S. M.; Barón, E.; González del Campo, M. del M.; Ferrer, J. M.; Berman-Riu, M.; García-Gasalla, M.; Aranda, M.; Borges, M.; de la Rica, R. Paper Biosensors for Detecting Elevated IL-6 Levels in Blood and Respiratory Samples from COVID-19 Patients. Sens Actuators B Chem 2021, 330, 129333. https://doi.org/10.1016/j.snb.2020.129333.
(76) Guo, H.; Yin, Z.; Namkoong, M.; Li, Y.; Nguyen, T.; Salcedo, E.; Arizpe, I.; Tian, L. Printed Ultrastable Bioplasmonic Microarrays for Point-of-Need Biosensing. ACS Appl Mater Interfaces 2022, 14 (8), 10729-10737. https://doi.org/10.1021/acsami. 1c24458.
(77) Yang, J.; Xiao, X.; Xia, L.; Li, G.; Shui, L. Microfluidic Magnetic Analyte Delivery Technique for Separation, Enrichment, and Fluorescence Detection of Ultratrace Biomarkers. Anal Chem 2021, 93 (23), 8273-8280. https://doi.org/10.1021/acs.analchem.1c01130.
(78) Jelliffe, R. W.; Schumitzky, A.; Bayard, D.; Fu, X.; Neely, M. Describing Assay Precision-Reciprocal of Variance Is Correct, Not C V Percent: Its Use Should Significantly Improve Laboratory Performance. Ther Drug Monit 2015, 37 (3), 389-394. https://doi.org/10.1097/FTD.0000000000000168.
(79) Platchek, M.; Lu, Q.; Tran, H.; Xie, W. Comparative Analysis of Multiple Immunoassays for Cytokine Profiling in Drug Discovery. SLAS Discovery 2020, 25 (10), 1197-1213. https://doi.org/10.1177/2472555220954389.
(80) Said, E. A.; Al-Reesi, I.; Al-Shizawi, N.; Jaju, S.; Al-Balushi, M. S.; Koh, C. Y.; Al-Jabri, A. A.; Jeyaseelan, L. Defining IL-6 Levels in Healthy Individuals: A Meta-analysis. J Med Virol 2021, 93 (6), 3915-3924. https://doi.org/10.1002/jmv.26654.
(81) Liu, G.; Jiang, C.; Lin, X.; Yang, Y. Point-of-Care Detection of Cytokines in Cytokine Storm Management and beyond: Significance and Challenges. View (Beijing, China) 2021, 2 (4), 20210003. https://doi.org/10.1002/VIW.20210003.
(82) Teijaro, J. R. Cytokine Storms in Infectious Diseases. Semin Immunopathol 2017, 39 (5), 501-503. https://doi.org/10.1007/s00281-017-0640-2.
(83) Tang, X. D.; Ji, T. T.; Dong, J. R.; Feng, H.; Chen, F. Q.; Chen, X.; Zhao, H. Y.; Chen, D. K.; Ma, W. T. Pathogenesis and Treatment of Cytokine Storm Induced by Infectious Discases. Int J Mol Sci 2021, 22 (23), 13009. https://doi.org/10.3390/ijms222313009.
(84) Huang, L.; Zhao, X.; Qi, Y.; Li, H.; Ye, G.; Liu, Y.; Zhang, Y.; Gou, J. Sepsis-Associated Severe Interleukin-6 Storm in Critical Coronavirus Disease 2019. Cell Mol Immunol 2020, 17 (10), 1092-1094. https://doi.org/10.1038/s41423-020-00522-6.
(85) Chaudhry, H.; Zhou, J.; Zhong, Y.; Ali, M. M.; McGuire, F.; Nagarkatti, P. S.; Nagarkatti, M. Role of Cytokines as a Double-Edged Sword in Sepsis. In Vivo 2013, 27 (6), 669-684. Kim, M. H.; Choi, J. H. An Update on Sepsis Biomarkers. Infect Chemother 2020, 52 (1), (86) 1. https://doi.org/10.3947/ic.2020.52.1.1.
(87) Sankar, V.; Webster, N. R. Clinical Application of Sepsis Biomarkers. J Anesth 2013, 27 (2), 269-283. https://doi.org/10.1007/s00540-012-1502-7.
(88) Guo, Y.; Xu, F.; Lu, T.; Duan, Z.; Zhang, Z. Interleukin-6 Signaling Pathway in Targeted Therapy for Cancer. Cancer Treat 2012, Rev 38 (7), 904-910. https://doi.org/10.1016/j.ctrv.2012.04.007.
(89) Wu, Y.; Wang, M.; Zhu, Y.; Lin, S. Scrum Interleukin-6 in the Diagnosis of Bacterial Infection in Cirrhotic Patients. Medicine 95 2016, (41), c5127. https://doi.org/10.1097/MD.0000000000005127.
(90) Thomas, X.; Hirschauer, C.; Troncy, J.; Assouline, D.; Joly, M. O.; Fiere, D.; Archimbaud, E. Scrum Interleukin-6 Levels in Adult Acute Myelogenous Leukemia: Relationship with Disease Characteristics and Outcome. Leuk Lymphoma 1997, 24 (3-4), 291-300. https://doi.org/10.3109/10428199709039016.
(91) Helle, M.; Boeije, L.; de Groot, E.; de Vos, A.; Aarden, L. Sensitive ELISA for Interleukin-6. J Immunol Methods 1991, 138 (1), 47-56. https://doi.org/10.1016/0022-1759(91)90063-L.

1. A pair of bioinks for printing a biosensor:
a first bioink being free of a crosslinking agent and comprising a scaffolding moiety and a biorecognition element for printing on a substrate; and
a second bioink comprising a crosslinking agent for printing on and crosslinking the first bioink to form the biosensor.

2. The pair of bioinks of claim 1, wherein the scaffolding moiety comprises BSA, collagen, gelatin, alginate, chitosan, or any combination thereof.

3. The pair of bioinks of claim 1, wherein the scaffolding moiety is present in an amount sufficient to achieve consistency in the morphology and circularity of the printed biosensor.

4. The pair of bioinks of claim 3, wherein the scaffolding moiety is present in an amount of less than about 2% by weight of the first bioink.

5. The pair of bioinks of claim 1, wherein the biorecognition element comprises an antibody or fragment thereof, a peptide, a polynucleotide, an aptamer, an aptamer-conjugated nanoparticle, a DNAzyme, a bacteriophage, or any combination thereof.

6. The pair of bioinks of claim 1, wherein the crosslinking agent comprises 1-ethyl-3-(3-dimethylaminopropyl) carbodiimide (EDC), N-hydroxysuccinimide (NHS), glutaraldehyde, N′,N′-dicyclohexyl carbodiimide (DCC), N,N′-diisopropyl carbodiimide (DIC), sulfo-NHS, thiophosgene, succinic anhydride, 3-mercaptopropyl trimethoxysilane (MPTMS), γ-maleimidobutyryloxy succinimide (GMBS), or any combination thereof.

7. The pair of bioinks of claim 6, wherein the crosslinking agent is present in an amount sufficient to crosslink the first bioink such that the biosensor is capable of withstanding intense washing with water and/or assay buffer.

8. The pair of bioinks of claim 7, wherein the crosslinking agent is present in an amount of about 2 mg/mL.

9. The pair of bioinks of claim 1, wherein the substrate comprises glass, a thermoplastic substrate, cyclic olefin copolymer, polystyrene, polycarbonate, thermoset, polydimethylsiloxane, fabric, paper, cellulose, or any combination thereof.

10. A biosensor made from the pair of bioinks of claim 1, wherein the first bioink is printed on the substrate and the second bioink is printed on the first bioink.

11. The biosensor of claim 10, comprising a plurality of layers of the first and second bioinks.

12. The biosensor of claim 11, comprising two or three layers of the first and second bioinks.

13. A microarray comprising a plurality of the biosensors of claim 10.

14. The microarray of claim 13, wherein at least one of the biosensors comprises a first biorecognition element and at least one other of the biosensors comprises a second biorecognition element.

15. A method of printing a biosensor from the pair of bioinks of claim 1, the method comprising:
printing the first bioink onto the substrate; and
printing the second bioink onto the first bioink.

16. The method of claim 15, further comprising printing a second layer of the first bioink onto the second bioink followed by printing the second bioink onto the second layer of the first bioink.

17. The method of claim 16, comprising an incubation step before printing the second layer.

18. The method of claim 15, wherein the first bioink is printed from a first nozzle and the second bioink is printed from a second nozzle.
